(19) United States
(12) Patent Application Publication
(10) Pub. No.: US 2022/0169646 A1
(43) Pub. Date: Jun. 2, 2022

(54) HETEROCYCLIC COMPOUNDS AND USES THEREOF

(71) Applicant: Nuvation Bio Inc., New York, NY (US)

(72) Inventors: Sarvajit CHAKRAVARTY, Edmond, OK (US); Son Minh PHAM, San Francisco, CA (US); Jayakanth KANKANALA, Plymouth, MN (US); Brahmam PUJALA, Greater Noida (IN); Sanjeev SONI, Noida (IN); Puja JAISWAL, Greater Noida (IN); Deepak PALVE, Noida (IN); Varun KUMAR, Noida (IN)

(21) Appl. No.: 17/594,298

(22) PCT Filed: Apr. 8, 2020
(86) PCT No.: PCT/US2020/027305

§ 371 Date: Oct. 8, 2021

Related U.S. Application Data
(60) Provisional application No. 62/831,672, filed on Apr. 9, 2019.

(51) Int. Cl. C07D 471/04; A61K 31/519; A61K 45/06; A61P 35/00

(52) U.S. Cl. CPC C07D 471/04; A61P 35/00; A61K 45/06; A61K 31/519

(57) ABSTRACT

Heterocyclic compounds as Wee1 inhibitors are provided. The compounds may find use as therapeutic agents for the treatment of diseases and may find particular use in oncology.

### CROSS REFERENCE TO RELATED APPLICATION

This application is a national stage application under 35 U.S.C. § 371 of International Application No. PCT/US2020/027305, filed internationally on Apr. 8, 2020, which claims priority to U.S. Provisional Application No. 62/831,672, filed on Apr. 9, 2019, the contents of which are incorporated herein by reference in their entirety.

### FIELD OF THE INVENTION

This disclosure relates generally to therapeutics engaged in inhibition of the DNA damage checkpoint kinase, Wee1, which potentiates genotoxic chemotherapies by abrogating cell-cycle arrest and proper DNA repair. The invention also provides pharmaceutically acceptable compositions comprising compounds of the present invention and methods of using said compositions in the treatment of diseases associated with this pathway.

### BACKGROUND OF THE INVENTION

Wee1 is a tyrosine kinase that phosphorylates and inactivates Cdc2 and is involved in G checkpoint signaling. More particularly, Wee1 is involved in G₂-M checkpoint signaling. Because p53 is a key regulator in the G checkpoint, p53-deficient tumors rely only on the G checkpoint after DNA damage. More particularly, because p53 is a key regulator in the G₁-S checkpoint, p53-deficient tumors rely only on the G₂-M checkpoint after DNA damage. Hence, such tumors are selectively sensitized to DNA-damaging agents by Wee1 inhibition.

Wee1 belongs to a family of protein kinases involved in the terminal phosphorylation and inactivation of cyclin-dependent kinase 1-bound cyclin B, resulting in G cell cycle arrest in response to DNA damage. Wee1 was first identified in fission yeast, where Wee1 deficiency resulted in premature mitotic entry and replication of smaller-sized yeast. It is the major kinase responsible for the inhibitory phosphorylation of the tyrosine.

Before cells undergo mitosis, they progress through a tightly controlled cascade of G₁-S, intra-S, and G₂-M checkpoints. Wee1 kinase has emerged as a key G₂-M checkpoint regulator. This tyrosine kinase negatively regulates entry into mitosis by catalyzing an inhibitory phosphorylation of Cdc2 (the human homolog of cyclin-dependent kinase 1 (CDK1) on tyrosine-15 (Y15). This results in inactivation of the Cdc2/cyclin B complex, which arrests cells in G₂-M, allowing for DNA repair. Such inhibition also occurs through Chk1-mediated inhibition of Cdc25 phosphatases, which remove the inhibitory phosphorylation on Cdc2. Thus, entry into mitosis rests on a balance between the opposing activities of Wee1 and Chk1/Cdc25. Wee1 inhibition is thus expected to abrogate G₂-M arrest and propel cells into premature mitosis, a hypothesis confirmed by studies documenting that Wee1 inhibition by either small molecule inhibitors or small interference RNA leads to premature entry into mitosis and consequent cell death through mitotic catastrophe or apoptosis. (S. Muller, J. Clinical. Oncology, 2015).

Recently, a few classes of Wee1 inhibitors have been disclosed. Among them is a selective inhibitor, AZD-1775 (1, 2-allyl-1-(6-(2-hydroxypropan-2-yl)pyridin-2-yl)-6-((4-(4methylpiperazin-1-yl)phenyl)amino)-1H-pyrazolo[3,4-d]pyrimidin-3(2H)-one). AZD-1775 exhibited antitumor activity in various preclinical studies as a monotherapy or in potentiating chemo- and radiotherapy, and is currently in phase I/II clinical trials.

Wee1 is highly expressed in several cancer types, including hepatocellular carcinoma, breast cancers, cervical cancers, lung cancers, squamous cell carcinoma, diffuse intrinsic pontine glioma (DIPG), glioblastoma, medulloblastoma, leukemia, melanoma, and ovarian cancers. (P. Reigan et al., Trends in Pharmacol. Sci., 2016).

There are few Wee1 inhibitors in clinical development. There is scope to improve Wee1 inhibitor selectivity and the properties of the inhibitors to permit targeting of specific cancer types.

### BRIEF SUMMARY OF THE INVENTION

In one aspect, provided is a compound of Formula (I):

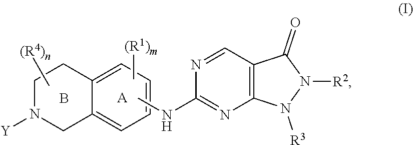

or a salt thereof, wherein Y, R¹, R², R³, R⁴, m and n are as detailed herein.

In some embodiments, the compound of Formula (I) or a salt thereof, is of the Formula (II) or (III), or a salt thereof as detailed herein.

In another aspect, provided is a method of treating cancer in an individual in need thereof comprising administering to the individual a therapeutically effective amount of a compound as detailed herein, such as a compound of Formula (I), (II) or (III) or a pharmaceutically acceptable salt thereof. Also provided is a method of inhibiting Wee1 in a cell, comprising administering a compound detailed herein, or a salt thereof, to the cell.

In another aspect, provided are pharmaceutical compositions comprising a compound detailed herein and a pharmaceutically acceptable carrier or excipient. Kits comprising a compound detailed herein or a salt thereof are also provided. A compound as detailed herein, or a salt thereof, is also provided for the manufacture of a medicament for the treatment of cancer.

### DETAILED DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like. It is understood that an alkylene can be linear or branched. Examples of branched alkylene include, but are not limited to, —CH(CH₃)CH₂—, —CH(CH₃)CH₂CH₂—, —C(CH₃)₂CH₂—, and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantyl, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

“Heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, and the like.

“Heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective dosage can be administered in one or more administrations. For purposes of this disclosure, an effective dosage of compound or a salt thereof, or pharmaceutical composition is an amount sufficient to accomplish prophylactic or therapeutic treatment either directly or indirectly. It is intended and understood that an effective dosage of a compound or salt thereof, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

In one aspect, provided is a compound of Formula (I):

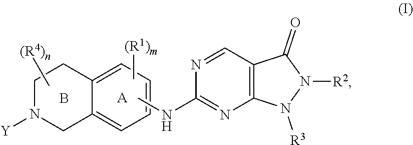

or a salt thereof, wherein:
Y is hydrogen or R⁴;
m is 0, 1, 2, or 3;
n is 0, 1, 2, 3, or 4;
R¹ is independently F, Cl, or methyl;
R² is independently C₁-C₆ alkyl, C₃-C₆ cycloalkyl or —(C₁-C₃ alkylene)CF₃;

### R³ is

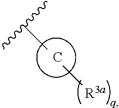

wherein:
ring C is a 5- or 6-membered heteroaryl or phenyl,
each R^{3a} is independently —CN, halogen, C₃-C₆ cycloalkyl, or —(C₁-C₆ alkylene)R′, wherein the C₁-C₆ alkylene is optionally substituted by oxo and wherein R′ is hydrogen, —CN, —NR¹⁹R²⁰, C₁-C₆ alkoxy, or —OH,
q is 0, 1, 2, 3, or 4;
   each R⁴ is independently oxo, C₁-C₆ alkyl, C₂-C₆ alkenyl, C₂-C₆ alkynyl, halogen, —C(O)R¹⁷, —C(O)OR¹⁷, —C(O)NR¹⁷R¹⁸, —CN, —Si(C₁-C₆ alkyl)₃, —OR¹⁷, —NR¹⁷R¹⁸, —OC(O)NR¹⁷R¹⁸, —NR¹⁷C(O)R¹⁸, —S(O)₂R¹⁷, —NR¹⁷S(O)₂R¹⁸, —S(O)₂NR¹⁷R¹⁸, C₃-C₆ cycloalkyl, 3- to 6-membered heterocyclyl, —(C₁-C₃ alkylene)CN, —(C₁-C₃ alkylene)OR¹⁷, —(C₁-C₃ alkylene)NR¹⁷R¹⁸, —(C₁-C₃ alkylene)CF₃, —(C₁-C₃ alkylene)C(O)R¹⁷, —(C₁-C₃ alkylene)C(O)NR¹⁷R¹⁸, —(C₁-C₃ alkylene)NR¹⁷C(O)R¹⁸, —(C₁-C₃ alkylene)S(O)₂R¹⁷, —(C₁-C₃ alkylene)NR¹⁷S(O)₂R¹⁸, —(C₁-C₃ alkylene)S(O)₂NR¹⁷R¹⁸, —(C₁-C₃ alkylene)(C₃-C₆ cycloalkyl) or —(C₁-C₃ alkylene)(3- to 6-membered heterocyclyl), wherein each R⁴ is independently optionally substituted by halogen, oxo, —OR¹⁹, —NR¹⁹R²⁰, or —C(O)R¹⁹,

or two R⁴, when bound to the same carbon, are taken together with the carbon to which they are attached to form a C₃-C₆ cycloalkyl or 3- to 6-membered heterocyclyl, each is optionally substituted by R¹⁹; and

each R¹⁷, R¹⁸, R¹⁹, and R²⁰ is independently hydrogen, C₃-C₆ cycloalkyl, 3-6 membered heterocyclyl or C₁-C₆ alkyl, each of which is optionally substituted by halogen, oxo or —OH, or R¹⁷ and R¹⁸ are taken together with the atom to which they attached to form a 3-6 membered heterocyclyl optionally substituted by halogen, oxo or —OH.

In some embodiments, provided is a compound of Formula (I):

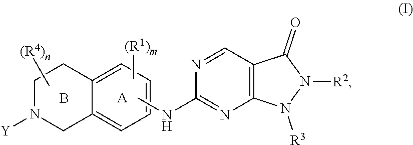

or a salt thereof, wherein:
Y is hydrogen or R⁴;
m is 0, 1, 2, or 3;
n is 0, 1, 2, 3, or 4;
R¹ is independently F, Cl, or methyl;
R² is independently C₁-C₆ alkyl, C₃-C₆ cycloalkyl or —(C₁-C₃ alkylene)CF₃;

### R³ is

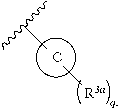

wherein:

ring C is a 5- or 6-membered heteroaryl or phenyl,

each R^{3a} is independently halogen, C₃-C₆ cycloalkyl, or —(C₁-C₆ alkylene)R′, wherein the C₁-C₆ alkylene is optionally substituted by oxo and wherein R′ is hydrogen, —CN, —NR¹⁹R²⁰, C₁-C₆ alkoxy, or —OH,

q is 0, 1, 2, 3, or 4;

each R⁴ is independently oxo, C₁-C₆ alkyl, C₂-C₆ alkenyl, C₂-C₆ alkynyl, halogen, —C(O)R¹⁷, —C(O)OR¹⁷, —C(O)NR¹⁷R¹⁸, —CN, —Si(C₁-C₆ alkyl)₃, —OR¹⁷, —NR¹⁷R¹⁸, —OC(O)NR¹⁷R¹⁸, —NR¹⁷C(O)R¹⁸, —S(O)₂R¹⁷, —NR¹⁷S(O)₂R¹⁸, —S(O)₂NR¹⁷R¹⁸, C₃-C₆ cycloalkyl, 3- to 6-membered heterocyclyl, —(C₁-C₃ alkylene)CN, —(C₁-C₃ alkylene)OR¹⁷, —(C₁-C₃ alkylene)NR¹⁷R¹⁸, —(C₁-C₃ alkylene)CF₃, —(C₁-C₃ alkylene)C(O)R¹⁷, —(C₁-C₃ alkylene)C(O)NR¹⁷R¹⁸, —(C₁-C₃ alkylene)NR¹⁷C(O)R¹⁸, —(C₁-C₃ alkylene)S(O)₂R¹⁷, —(C₁-C₃ alkylene)NR¹⁷S(O)₂R¹⁸, —(C₁-C₃ alkylene)S(O)₂NR¹⁷R¹⁸, —(C₁-C₃ alkylene)(C₃-C₆ cycloalkyl) or —(C₁-C₃ alkylene)(3- to 6-membered heterocyclyl), wherein each R⁴ is independently optionally substituted by halogen, oxo, —OR¹⁹, —NR¹⁹R²⁰, or —C(O)R¹⁹,

or two R⁴, when bound to the same carbon, are taken together with the carbon to which they are attached to form a C₃-C₆ cycloalkyl or 3- to 6-membered heterocyclyl, each is optionally substituted by R¹⁹; and

each R¹⁷, R¹⁸, R¹⁹, and R²⁰ is independently hydrogen, C₃-C₆ cycloalkyl, 3-6 membered heterocyclyl or C₁-C₆ alkyl, each of which is optionally substituted by halogen, oxo or —OH,

or R¹⁷ and R¹⁸ are taken together with the atom to which they attached to form a 3-6 membered heterocyclyl optionally substituted by halogen, oxo or —OH.

In some embodiments of a compound of Formula (I), the compound has one or more of the following features:
1. when C ring is pyridinyl substituted with

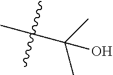 [SMILES: *C(C)(C)O]

or an imidazolyl substituted with

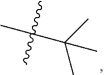 [SMILES: *C(C)(C)C]

then A ring is substituted with R¹;
2. when R² is isopropyl and A, B, Y, R¹, R⁴, m and n together is

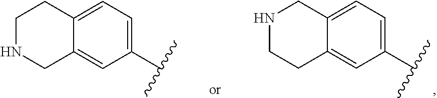 [SMILES: *c1ccc2c(c1)CNCC2 ; *c1ccc2c(c1)CCNC2]

then phenyl of C ring is not substituted by halogen; and
3. when R³ is

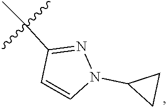 [SMILES: *c1ccn(C2CC2)n1]

then A, B, Y, R¹, R⁴, m and n together is not

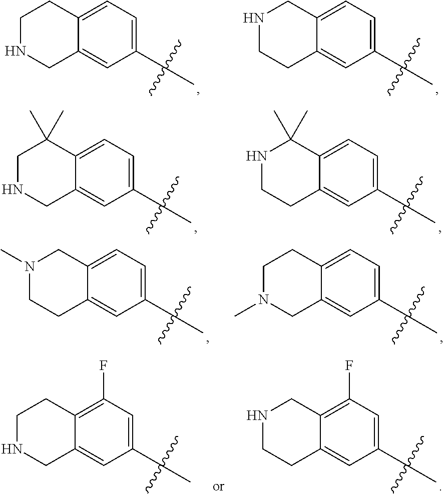

In some embodiments of a compound of Formula (I), the compound is of Formula (II):

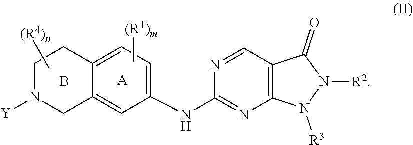

In some embodiments of a compound of Formula (I), the compound is of Formula (III):

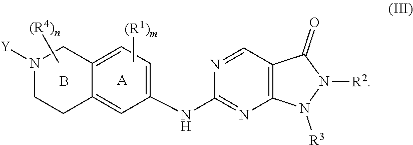

In some embodiments of a compound of Formula (I), R² is C₁-C₆ alkyl, such as methyl, ethyl, n-propyl, isopropyl, n-butyl, t-butyl, isobutyl, or sec-butyl. In some embodiments, R² is isopropyl or ethyl. In some embodiments, R² is isopropyl. In some embodiments, R² is ethyl. In some embodiments, R² is C₃₋₆ cycloalkyl, such as cyclopropyl, cyclobutyl, cyclopentyl, or cyclohexyl. In some embodiments, R² is cyclopropyl. In some embodiments, R² is —(C₁-C₃ alkylene)CF₃. In some embodiments, R² is —CH₂CF₃. In some embodiments, R² is selected from the group consisting of isopropyl, ethyl, cyclopropyl, and —CH₂CF₃.

In some embodiments of a compound of Formula (I), ring C is 5- or 6-membered heteroaryl, such as pyridinyl, pyrazinyl, pyridazinyl, primidinyl, triazinyl, pyrrolyl, pyrazolyl, imidazolyl, triazolyl, tetrazolyl, oxazolyl, thiazolyl, thiazolyl, or furanyl. In some embodiment, ring C is a 6-membered heteroaryl, such as pyridinyl, pyrazinyl, pyridazinyl, or pyrimidinyl. In some embodiments, ring C is a 5-membered heteroaryl, such as triazinyl, pyrrolyl, pyrazolyl, imidazolyl, triazolyl, tetrazolyl, oxazolyl, thiazolyl, thiazolyl, or furanyl. In some embodiments, ring C is phenyl. In some embodiments, ring C is selected from the group consisting of:

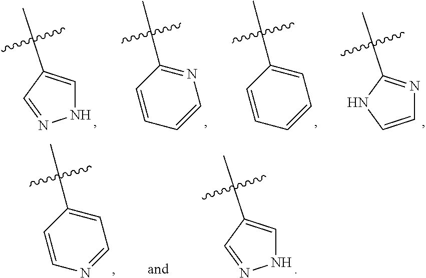 [SMILES: *c1cn[nH]c1 ; *c1ccccn1 ; *c1ccccc1 ; *c1ncc[nH]1 ; *c1ccncc1 ; *c1cn[nH]c1]

In some embodiments, ring C is

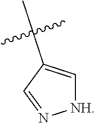 [SMILES: *c1cn[nH]c1]

In some embodiments, ring C is

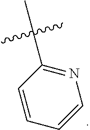 [SMILES: *c1ccccn1]

In some embodiments, ring C is

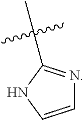 [SMILES: *c1ncc[nH]1]

In some embodiments, ring C is

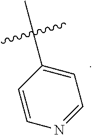 [SMILES: *c1ccncc1]

In some embodiments, ring C is

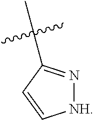 [SMILES: *c1cc[nH]n1]

In some embodiments of a compound of Formula (I), R^{3a} is C₃-C₆ cycloalkyl, such as cyclopropyl, cyclobutyl, cyclopentyl, or cyclohexy. In some embodiments, R^{3a} is cyclopropy. In some embodiments, R³ is —(C₁-C₆ alkylene)R′, wherein the C₁-C₆ alkylene is optionally substituted by oxo and wherein R′ is hydrogen, —CN, —NR¹⁹R²⁰, C₁-C₆ alkoxy, or —OH. In some embodiments, R^{3a} is —(C₁-C₆ alkylene)R′, wherein the C₁-C₆ alkylene is optionally substituted by oxo and wherein R′ is hydrogen. In some embodiments, R^{3a} is

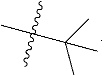 [SMILES: *C(C)(C)C]

In some embodiments, R^{3a} is —(C₁-C₆ alkylene)R′, wherein the C₁-C₆ alkylene is optionally substituted by oxo and wherein R′ is —CN. In some embodiments, R^{3a} is

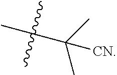 [SMILES: *C(C)(C)C#N]

In some embodiments, R^{3a} is —(C₁-C₆ alkylene)R′, wherein the C₁-C₆ alkylene is optionally substituted by oxo and wherein R′ is —NR¹⁹R²⁰. In some embodiments, R^{3a} is

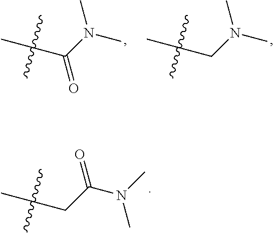

In some embodiments, R^{3a} is

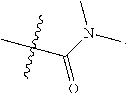 [SMILES: *C(=O)N(C)C]

In some embodiments, R^{3a} is

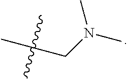 [SMILES: *CN(C)C]

In some embodiments, R^{3a} is

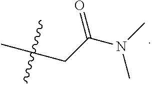 [SMILES: *CC(=O)N(C)C]

In some embodiments, R^{3a} is —(C₁-C₆ alkylene)R′, wherein the C₁-C₆ alkylene is optionally substituted by oxo and wherein R′ is C₁-C₆ alkoxy. In some embodiments, R^{3a} is

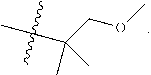 [SMILES: *C(C)(C)COC]

In some embodiments, R^{3a} is halogen such as fluoro, chloro, bromo, or iodo. In some embodiments, R^{3a} is fluoro. In some embodiments, R^{3a} is —(C₁-C₆ alkylene)R′, wherein the C₁-C₆ alkylene is optionally substituted by oxo and wherein R′ is —OH. In some embodiments, R^{3a} is

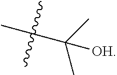 [SMILES: *C(C)(C)O]

In some embodiments, R^{3a} is selected from the group consisting of:

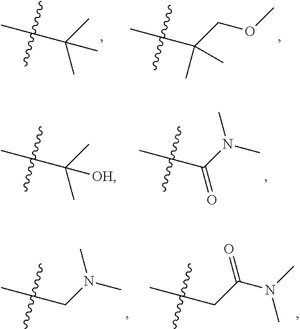

fluoro, and cyclopropyl.

In some embodiments of a compound of Formula (I), q is 0. In some embodiments, q is 1. In some embodiments, q is 2. In some embodiments, q is 3. In some embodiments, q is 4. In some embodiments, q is 0, 1, 2, or 3. In some embodiments, q is 0, 1, or 2. In some embodiments, q is 0 or 1.

In some embodiments of a compound of Formula (I), R³ is selected from the group consisting of:

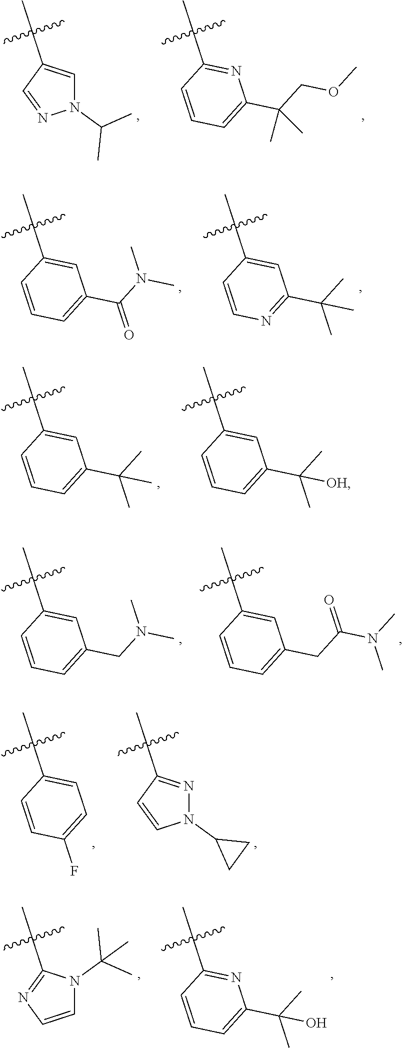

and

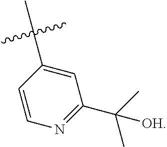 [SMILES: *c1ccnc(C(C)(C)O)c1]

In some embodiments, R³ is

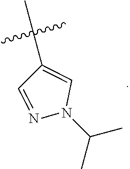 [SMILES: *c1cnn(C(C)C)c1]

In some embodiments, R³ is

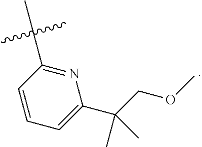 [SMILES: *c1cccc(C(C)(C)COC)n1]

In some embodiments, R³ is

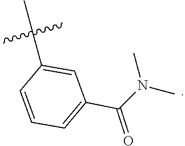 [SMILES: *c1cccc(C(=O)N(C)C)c1]

In some embodiments, R³ is

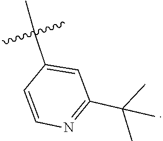 [SMILES: *c1ccnc(C(C)(C)C)c1]

In some embodiments, R³ is

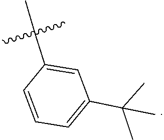 [SMILES: *c1cccc(C(C)(C)C)c1]

In some embodiments, R³ is

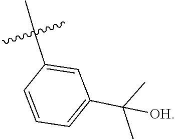 [SMILES: *c1cccc(C(C)(C)O)c1]

In some embodiments, R³ is

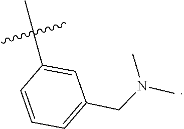 [SMILES: *c1cccc(CN(C)C)c1]

In some embodiments, R³ is

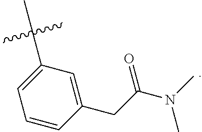 [SMILES: *c1cccc(CC(=O)N(C)C)c1]

In some embodiments, R³ is

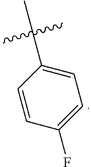 [SMILES: *c1ccc(F)cc1]

In some embodiments, R³ is

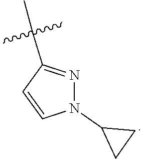 [SMILES: *c1ccn(C2CC2)n1]

In some embodiments, R³ is

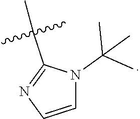 [SMILES: *c1nccn1C(C)(C)C]

In some embodiments, R³ is

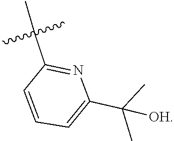 [SMILES: *c1cccc(C(C)(C)O)n1]

In some embodiments, R³ is

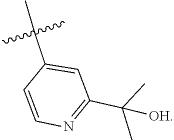 [SMILES: *c1ccnc(C(C)(C)O)c1]

In some embodiments of a compound of Formula (I), m is 0. In some embodiments, m is 1. In some embodiments, m is 2. In some embodiments, m is 3. In some embodiments, m is 0, 1, or 2. In some embodiments, m is 0 or 1.

In some embodiments of a compound of Formula (I), R¹ is F. In some embodiments, R¹ is Cl. In some embodiments R¹ is methyl. In some embodiments, m is 1 and R¹ is F.

In some embodiments of a compound of Formula (I), n is 0. In some embodiments, n is 1. In some embodiments, n is 2. In some embodiments, n is 3. In some embodiments, n is 4. In some embodiments, n is 0, 1, 2, or 3. In some embodiments, n is 0, 1, or 2. In some embodiments, n is 0 or 1.

In some embodiments of a compound of Formula (I), each R⁴ is independently C₁-C₆ alkyl, or two R⁴, when bound to the same carbon, are taken together with the carbon or carbons to which they are attached to form a C₃-C₆ cycloalkyl. In some embodiments, each R⁴ is independently C₁-C₆ alkyl, such as methyl, ethyl, n-propyl, isopropyl, n-butyl, t-butyl, isobutyl, or sec-butyl. In some embodiments, n is 1 and R⁴ is C₁-C₆ alkyl. In some embodiments, n is 2 and each R⁴ is independently C₁-C₆ alkyl. In some embodiments, n is 2 and each R⁴ is methyl. In some embodiments, n is 2 and two R⁴, when bound to the same carbon, are taken together with the carbon to which they are attached to form a C3-C₆ cycloalkyl. In some embodiments, n is 2 and two R⁴, when bound to the same carbon, are taken together with the carbon to which they are attached to form a cyclopropyl.

In some embodiments of a compound of Formula (I), Y is hydrogen. In some embodiments, Y is R⁴. In some embodiments, Y is C₁-C₆ alkyl, C₂-C₆ alkenyl, C₂-C₆ alkynyl, C₃-C₆ cycloalkyl, or 3- to 6-membered heterocyclyl. In some embodiments, Y is C₁-C₆ alkyl, such as methyl, ethyl, n-propyl, isopropyl, n-butyl, t-butyl, isobutyl, or sec-butyl. In some embodiments, Y is isopropyl.

In some embodiments of a compound of Formula (I), ring A, ring B, Y, R¹ and R⁴ together are taken together to form a moiety selected from the group consisting of:

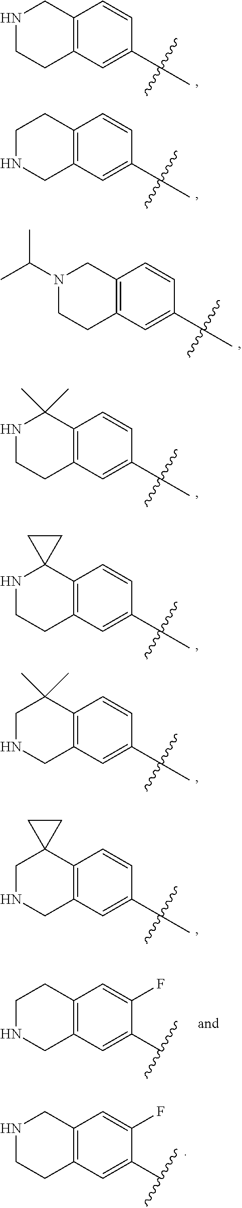

In some embodiments, ring A, ring B, Y, R¹ and R⁴ together are taken together to form

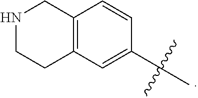 [SMILES: *c1ccc2c(c1)CCNC2]

In some embodiments, ring A, ring B, Y, R¹ and R⁴ together are taken together to form

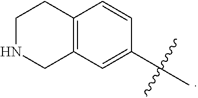 [SMILES: *c1ccc2c(c1)CNCC2]

In some embodiments, ring A, ring B, Y, R¹ and R⁴ together are taken together to form

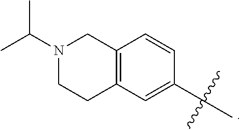 [SMILES: *c1ccc2c(c1)CCN(C(C)C)C2]

In some embodiments, ring A, ring B, Y, R¹ and R⁴ together are taken together to form

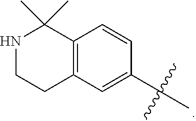 [SMILES: *c1ccc2c(c1)CCNC2(C)C]

In some embodiments, ring A, ring B, Y, R¹ and R⁴ together are taken together to form

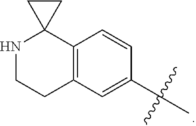 [SMILES: *c1ccc2c(c1)CCNC21CC1]

In some embodiments, ring A, ring B, Y, R¹ and R⁴ together are taken together to form

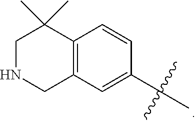 [SMILES: *c1ccc2c(c1)CNCC2(C)C]

In some embodiments, ring A, ring B, Y, R¹ and R⁴ together are taken together to form

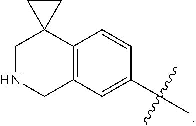 [SMILES: *c1ccc2c(c1)CNCC21CC1]

In some embodiments, ring A, ring B, Y, R¹ and R⁴ together are taken together to form

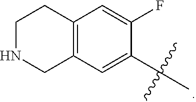 [SMILES: *c1cc2c(cc1F)CCNC2]

In some embodiments, ring A, ring B, Y, R¹ and R⁴ together are taken together to form

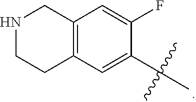 [SMILES: *c1cc2c(cc1F)CNCC2]

In some embodiments of a compound of Formula (I), the compound has one or more of the following features:

### (I) R² is

(1) C₁-C₆ alkyl, such as isopropyl or ethyl,
(2) C₃-C₆ cycloalkyl, such as cyclopropyl, or
(3) —(C₁-C₃ alkylene)CF₃, such as —CH₂CF₃;

### (II) R³ is

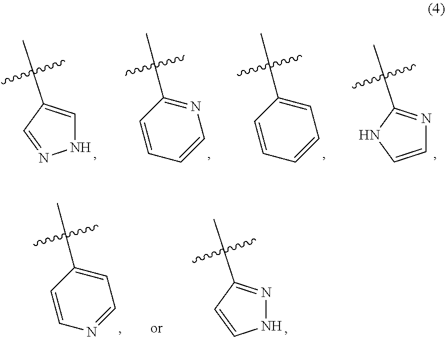 [SMILES: *c1cn[nH]c1 ; *c1ccccn1 ; *c1ccccc1 ; *c1ncc[nH]1 ; *c1ccncc1 ; *c1cc[nH]n1]

each of which is optionally substituted by one, two, three, or four R^{3a}, or

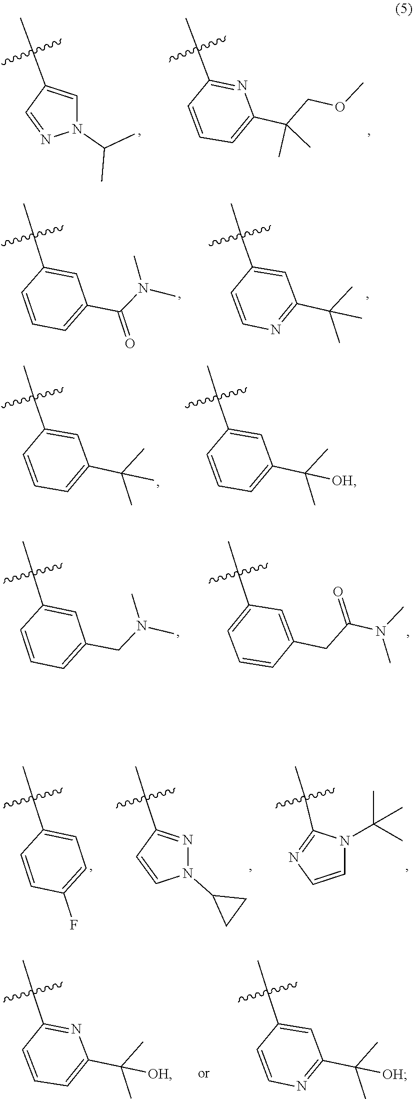

(III) ring A, ring B, R¹, and R⁴ are taken together to form a moiety selected from the group consisting of:

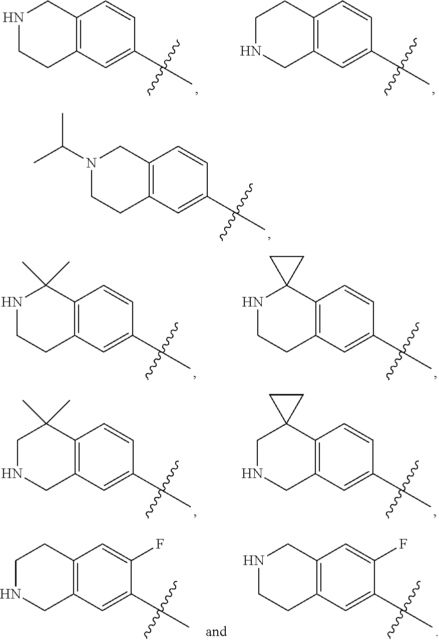

In some embodiments, (1) applies. In some embodiments, (2) applies. In some embodiments, (3) applies. In some embodiments, (4) applies. In some embodiments, (5) applies. In some embodiments, (III) applies. In some embodiments, (I) and (4) apply. In some embodiments, (I) and (5) apply. In some embodiments, (1) and (4) apply. In some embodiments, (1) and (5) apply. In some embodiments, (2) and (4) apply. In some embodiments, (2) and (5) apply. In some embodiments, (3) and (4) apply. In some embodiments, (3) and (5) apply. In some embodiments, (I) and (III) apply. In some embodiments, (1) and (III) apply. In some embodiments, (2) and (III) apply. In some embodiments, (3) and (III) apply. In some embodiments, (4) and (III) apply. In some embodiments, (5) and (III) apply. In some embodiments, (I), (4), and (III) apply. In some embodiments, (I), (5), and (III) apply. In some embodiments, (1), (4), and (III) apply. In some embodiments, (1), (5), and (III) apply. In some embodiments, (2), (4), and (III) apply. In some embodiments, (2), (5), and (III) apply. In some embodiments, (3), (4), and (III) apply. In some embodiments, (3), (5), and (III) apply.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of Formula (I) may be combined with every description, variation, embodiment or aspect of R², R³, R⁴, m, n, and Y the same as if each and every combination were specifically and individually listed. It is also understood that all descriptions, variations, embodiments or aspects of Formula (I), where applicable, apply equally to other formulae detailed herein, and are equally described, the same as if each and every description, variation, embodiment or aspect were separately and individually listed for all formulae. For example, all descriptions, variations, embodiments or aspects of formula (I), where applicable, apply equally to any of formulae as detailed herein, such as Formula (II) and Formula (III) and are equally described, the same as if each and every description, variation, embodiment or aspect were separately and individually listed for all formulae.

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds are listed in Table 1.

TABLE 1

| Compound | |
|---|---|
| No. | Structure |
| 1.1 | 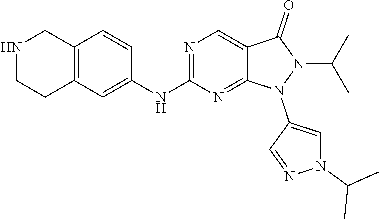 [SMILES: CC(C)n1cc(-n2c3nc(Nc4ccc5c(c4)CCNC5)ncc3c(=O)n2C(C)C)cn1] |
| 1.2 | 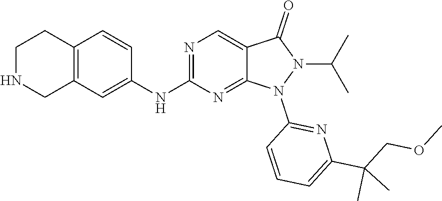 [SMILES: COCC(C)(C)c1cccc(-n2c3nc(Nc4ccc5c(c4)CNCC5)ncc3c(=O)n2C(C)C)n1] |
| 1.3 | 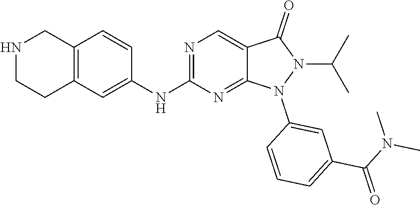 [SMILES: CC(C)n1c(=O)c2cnc(Nc3ccc4c(c3)CCNC4)nc2n1-c1cccc(C(=O)N(C)C)c1] |
| 1.4 | 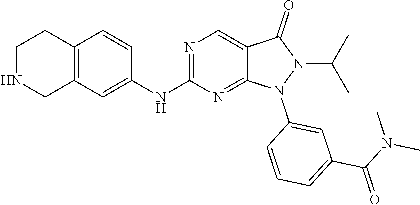 [SMILES: CC(C)n1c(=O)c2cnc(Nc3ccc4c(c3)CNCC4)nc2n1-c1cccc(C(=O)N(C)C)c1] |
| 1.5 | 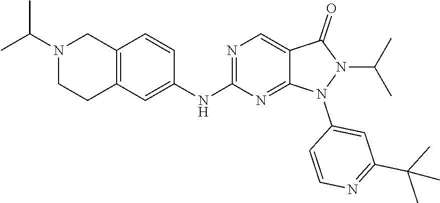 [SMILES: CC(C)N1CCc2cc(Nc3ncc4c(=O)n(C(C)C)n(-c5ccnc(C(C)(C)C)c5)c4n3)ccc2C1] |
| 1.6 | 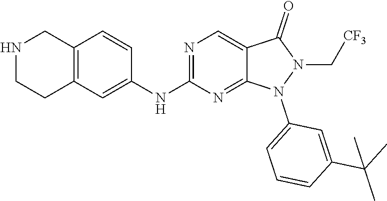 [SMILES: CC(C)(C)c1cccc(-n2c3nc(Nc4ccc5c(c4)CCNC5)ncc3c(=O)n2CC(F)(F)F)c1] |
| 1.7 | 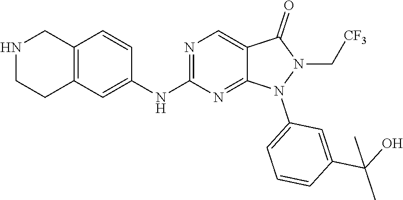 [SMILES: CC(C)(O)c1cccc(-n2c3nc(Nc4ccc5c(c4)CCNC5)ncc3c(=O)n2CC(F)(F)F)c1] |
| 1.8 | 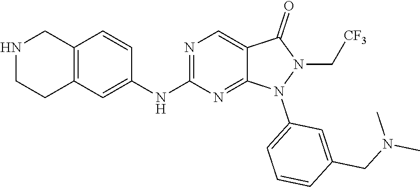 [SMILES: CN(C)Cc1cccc(-n2c3nc(Nc4ccc5c(c4)CCNC5)ncc3c(=O)n2CC(F)(F)F)c1] |
| 1.9 | 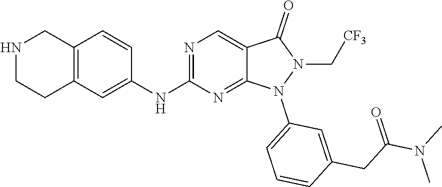 [SMILES: CN(C)C(=O)Cc1cccc(-n2c3nc(Nc4ccc5c(c4)CCNC5)ncc3c(=O)n2CC(F)(F)F)c1] |
| 1.10 | 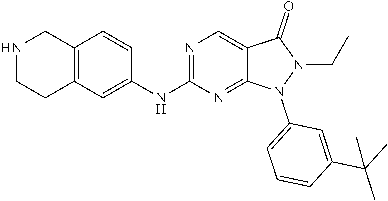 [SMILES: CCn1c(=O)c2cnc(Nc3ccc4c(c3)CCNC4)nc2n1-c1cccc(C(C)(C)C)c1] |
| 1.12 | 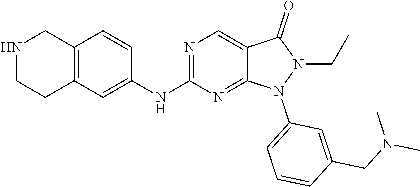 [SMILES: CCn1c(=O)c2cnc(Nc3ccc4c(c3)CCNC4)nc2n1-c1cccc(CN(C)C)c1] |
| 1.13 | 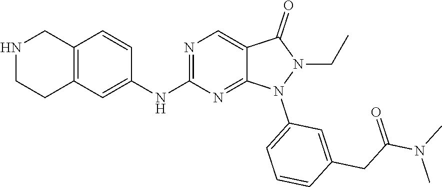 [SMILES: CCn1c(=O)c2cnc(Nc3ccc4c(c3)CCNC4)nc2n1-c1cccc(CC(=O)N(C)C)c1] |
| 1.14 | 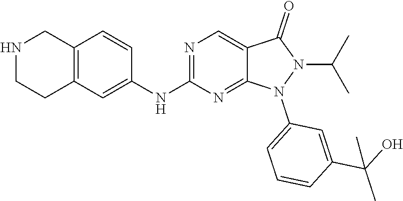 [SMILES: CC(C)n1c(=O)c2cnc(Nc3ccc4c(c3)CCNC4)nc2n1-c1cccc(C(C)(C)O)c1] |
| 1.15 | 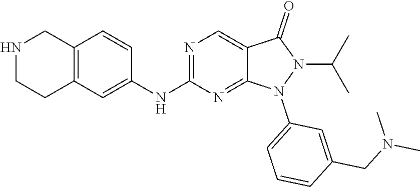 [SMILES: CC(C)n1c(=O)c2cnc(Nc3ccc4c(c3)CCNC4)nc2n1-c1cccc(CN(C)C)c1] |
| 1.16 | 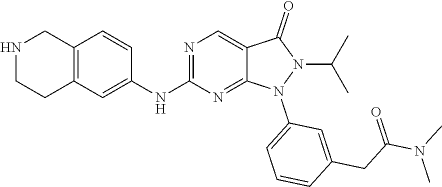 [SMILES: CC(C)n1c(=O)c2cnc(Nc3ccc4c(c3)CCNC4)nc2n1-c1cccc(CC(=O)N(C)C)c1] |
| 1.17 | 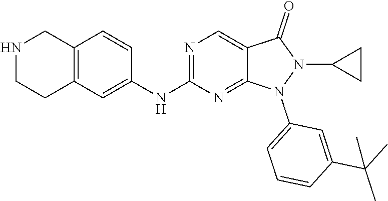 [SMILES: CC(C)(C)c1cccc(-n2c3nc(Nc4ccc5c(c4)CCNC5)ncc3c(=O)n2C2CC2)c1] |
| 1.18 | 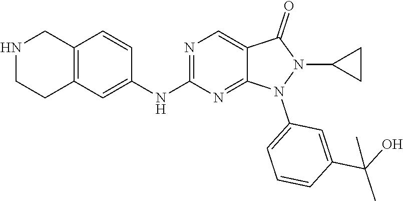 [SMILES: CC(C)(O)c1cccc(-n2c3nc(Nc4ccc5c(c4)CCNC5)ncc3c(=O)n2C2CC2)c1] |
| 1.19 | 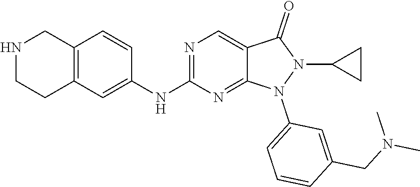 [SMILES: CN(C)Cc1cccc(-n2c3nc(Nc4ccc5c(c4)CCNC5)ncc3c(=O)n2C2CC2)c1] |
| 1.20 | 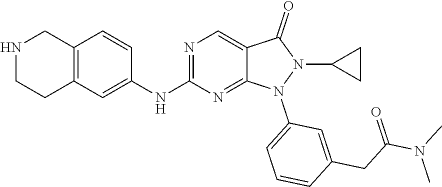 [SMILES: CN(C)C(=O)Cc1cccc(-n2c3nc(Nc4ccc5c(c4)CCNC5)ncc3c(=O)n2C2CC2)c1] |
| 1.21 | 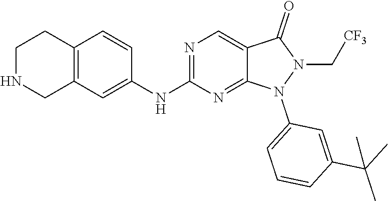 [SMILES: CC(C)(C)c1cccc(-n2c3nc(Nc4ccc5c(c4)CNCC5)ncc3c(=O)n2CC(F)(F)F)c1] |
| 1.22 | 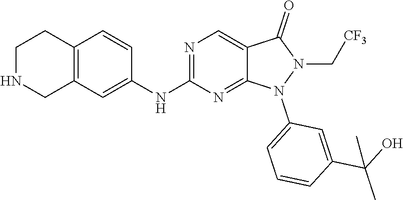 [SMILES: CC(C)(O)c1cccc(-n2c3nc(Nc4ccc5c(c4)CNCC5)ncc3c(=O)n2CC(F)(F)F)c1] |
| 1.23 | 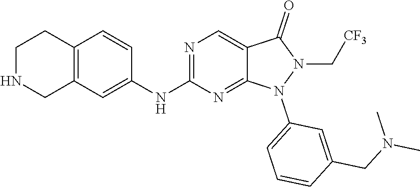 [SMILES: CN(C)Cc1cccc(-n2c3nc(Nc4ccc5c(c4)CNCC5)ncc3c(=O)n2CC(F)(F)F)c1] |
| 1.24 | 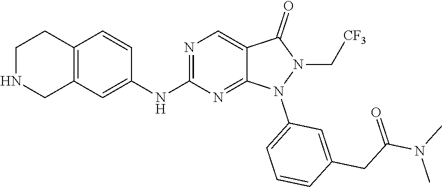 [SMILES: CN(C)C(=O)Cc1cccc(-n2c3nc(Nc4ccc5c(c4)CNCC5)ncc3c(=O)n2CC(F)(F)F)c1] |
| 1.25 | 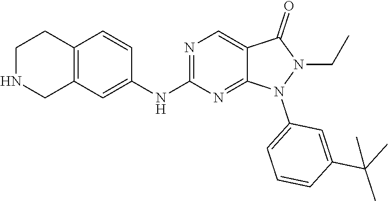 [SMILES: CCn1c(=O)c2cnc(Nc3ccc4c(c3)CNCC4)nc2n1-c1cccc(C(C)(C)C)c1] |
| 1.27 | 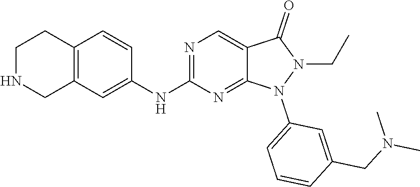 [SMILES: CCn1c(=O)c2cnc(Nc3ccc4c(c3)CNCC4)nc2n1-c1cccc(CN(C)C)c1] |
| 1.28 | 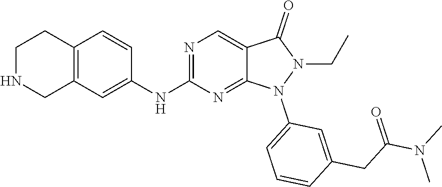 [SMILES: CCn1c(=O)c2cnc(Nc3ccc4c(c3)CNCC4)nc2n1-c1cccc(CC(=O)N(C)C)c1] |
| 1.30 | 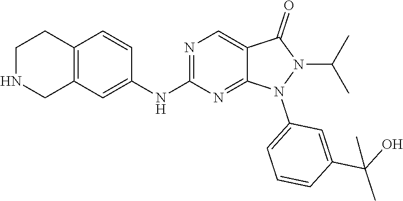 [SMILES: CC(C)n1c(=O)c2cnc(Nc3ccc4c(c3)CNCC4)nc2n1-c1cccc(C(C)(C)O)c1] |
| 1.31 | 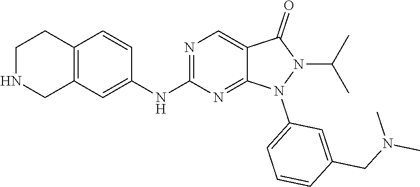 [SMILES: CC(C)n1c(=O)c2cnc(Nc3ccc4c(c3)CNCC4)nc2n1-c1cccc(CN(C)C)c1] |
| 1.32 | 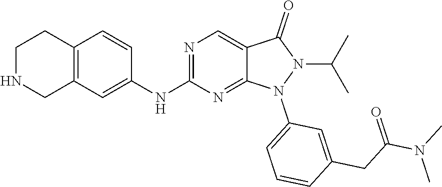 [SMILES: CC(C)n1c(=O)c2cnc(Nc3ccc4c(c3)CNCC4)nc2n1-c1cccc(CC(=O)N(C)C)c1] |
| 1.33 | 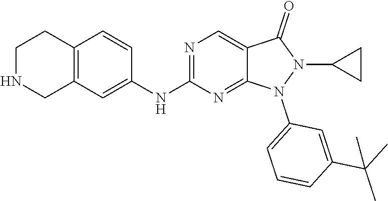 [SMILES: CC(C)(C)c1cccc(-n2c3nc(Nc4ccc5c(c4)CNCC5)ncc3c(=O)n2C2CC2)c1] |
| 1.34 | 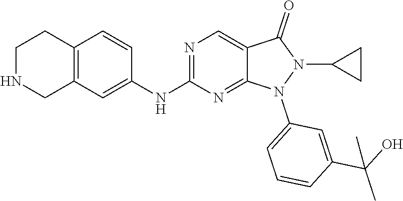 [SMILES: CC(C)(O)c1cccc(-n2c3nc(Nc4ccc5c(c4)CNCC5)ncc3c(=O)n2C2CC2)c1] |
| 1.35 | 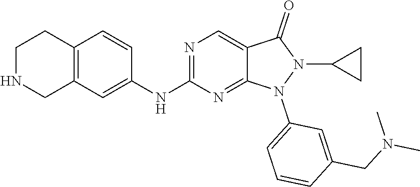 [SMILES: CN(C)Cc1cccc(-n2c3nc(Nc4ccc5c(c4)CNCC5)ncc3c(=O)n2C2CC2)c1] |
| 1.36 | 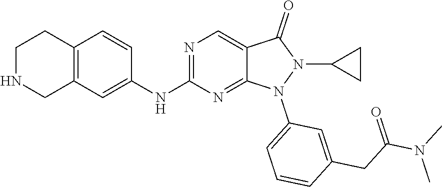 [SMILES: CN(C)C(=O)Cc1cccc(-n2c3nc(Nc4ccc5c(c4)CNCC5)ncc3c(=O)n2C2CC2)c1] |
| 1.37 | 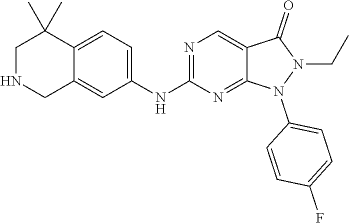 [SMILES: CCn1c(=O)c2cnc(Nc3ccc4c(c3)CNCC4(C)C)nc2n1-c1ccc(F)cc1] |
| 1.38 | 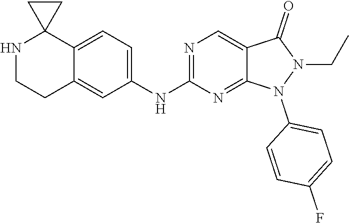 [SMILES: CCn1c(=O)c2cnc(Nc3ccc4c(c3)CCNC43CC3)nc2n1-c1ccc(F)cc1] |
| 1.39 | 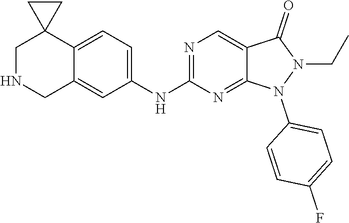 [SMILES: CCn1c(=O)c2cnc(Nc3ccc4c(c3)CNCC43CC3)nc2n1-c1ccc(F)cc1] |
| 1.40 | 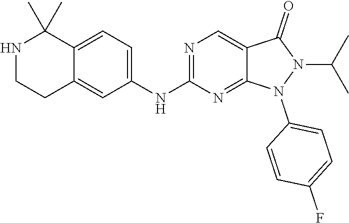 [SMILES: CC(C)n1c(=O)c2cnc(Nc3ccc4c(c3)CCNC4(C)C)nc2n1-c1ccc(F)cc1] |
| 1.41 | 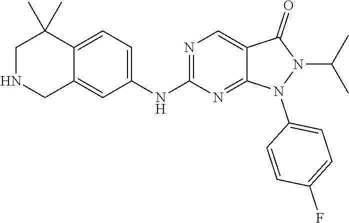 [SMILES: CC(C)n1c(=O)c2cnc(Nc3ccc4c(c3)CNCC4(C)C)nc2n1-c1ccc(F)cc1] |
| 1.42 | 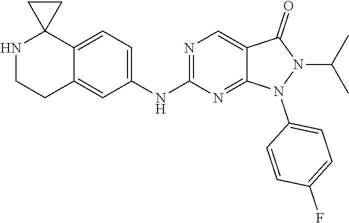 [SMILES: CC(C)n1c(=O)c2cnc(Nc3ccc4c(c3)CCNC43CC3)nc2n1-c1ccc(F)cc1] |
| 1.43 | 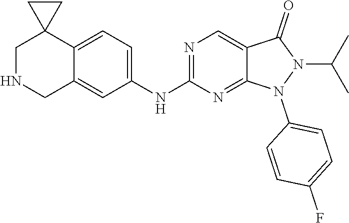 [SMILES: CC(C)n1c(=O)c2cnc(Nc3ccc4c(c3)CNCC43CC3)nc2n1-c1ccc(F)cc1] |
| 1.44 | 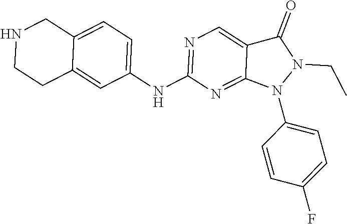 [SMILES: CCn1c(=O)c2cnc(Nc3ccc4c(c3)CCNC4)nc2n1-c1ccc(F)cc1] |
| 1.45 | 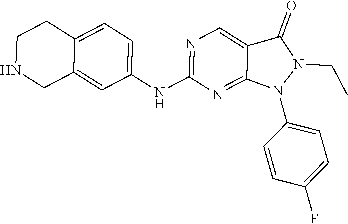 [SMILES: CCn1c(=O)c2cnc(Nc3ccc4c(c3)CNCC4)nc2n1-c1ccc(F)cc1] |
| 1.46 | 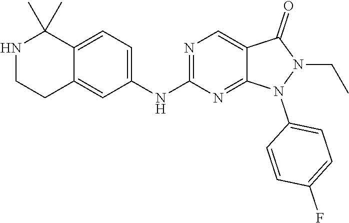 [SMILES: CCn1c(=O)c2cnc(Nc3ccc4c(c3)CCNC4(C)C)nc2n1-c1ccc(F)cc1] |
| 1.47 | 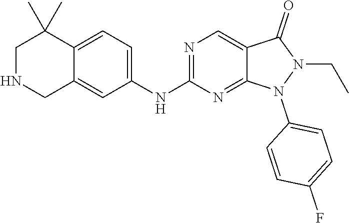 [SMILES: CCn1c(=O)c2cnc(Nc3ccc4c(c3)CNCC4(C)C)nc2n1-c1ccc(F)cc1] |
| 1.48 | 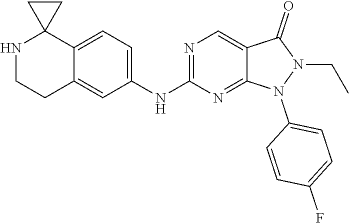 [SMILES: CCn1c(=O)c2cnc(Nc3ccc4c(c3)CCNC43CC3)nc2n1-c1ccc(F)cc1] |
| 1.49 | 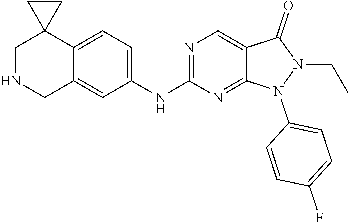 [SMILES: CCn1c(=O)c2cnc(Nc3ccc4c(c3)CNCC43CC3)nc2n1-c1ccc(F)cc1] |
| 1.50 | 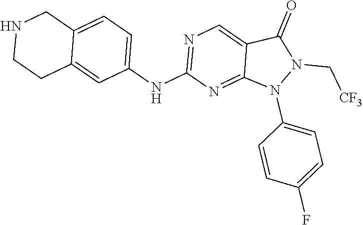 [SMILES: O=c1c2cnc(Nc3ccc4c(c3)CCNC4)nc2n(-c2ccc(F)cc2)n1CC(F)(F)F] |
| 1.51 | 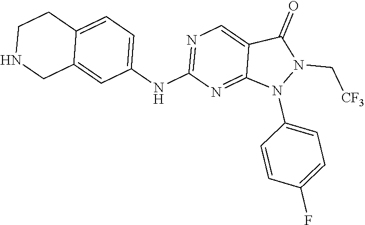 [SMILES: O=c1c2cnc(Nc3ccc4c(c3)CNCC4)nc2n(-c2ccc(F)cc2)n1CC(F)(F)F] |
| 1.52 | 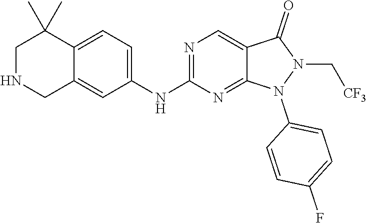 [SMILES: CC1(C)CNCc2cc(Nc3ncc4c(=O)n(CC(F)(F)F)n(-c5ccc(F)cc5)c4n3)ccc21] |
| 1.53 | 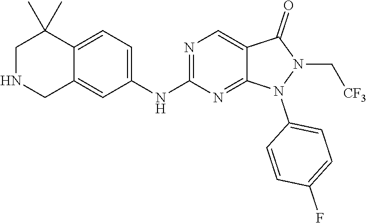 [SMILES: CC1(C)CNCc2cc(Nc3ncc4c(=O)n(CC(F)(F)F)n(-c5ccc(F)cc5)c4n3)ccc21] |
| 1.54 | 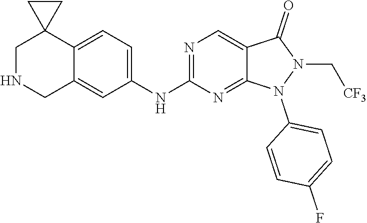 [SMILES: O=c1c2cnc(Nc3ccc4c(c3)CNCC43CC3)nc2n(-c2ccc(F)cc2)n1CC(F)(F)F] |
| 1.55 | 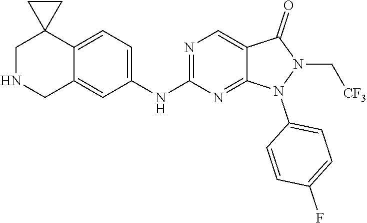 [SMILES: O=c1c2cnc(Nc3ccc4c(c3)CNCC43CC3)nc2n(-c2ccc(F)cc2)n1CC(F)(F)F] |
| 1.56 | 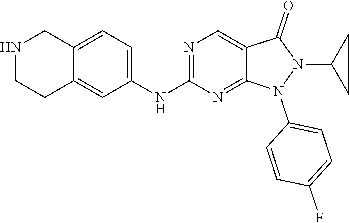 [SMILES: O=c1c2cnc(Nc3ccc4c(c3)CCNC4)nc2n(-c2ccc(F)cc2)n1C1CC1] |
| 1.57 | 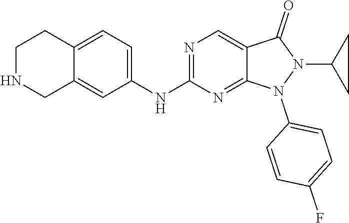 [SMILES: O=c1c2cnc(Nc3ccc4c(c3)CNCC4)nc2n(-c2ccc(F)cc2)n1C1CC1] |
| 1.58 | 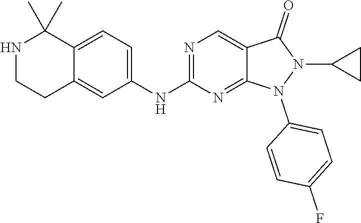 [SMILES: CC1(C)NCCc2cc(Nc3ncc4c(=O)n(C5CC5)n(-c5ccc(F)cc5)c4n3)ccc21] |
| 1.59 | 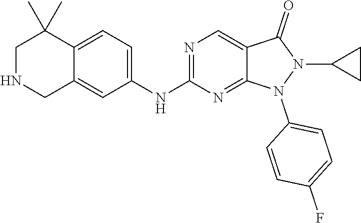 [SMILES: CC1(C)CNCc2cc(Nc3ncc4c(=O)n(C5CC5)n(-c5ccc(F)cc5)c4n3)ccc21] |
| 1.60 | 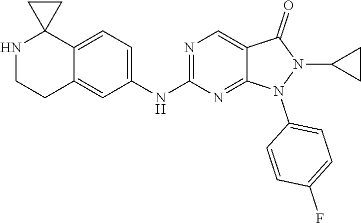 [SMILES: O=c1c2cnc(Nc3ccc4c(c3)CCNC43CC3)nc2n(-c2ccc(F)cc2)n1C1CC1] |
| 1.61 | 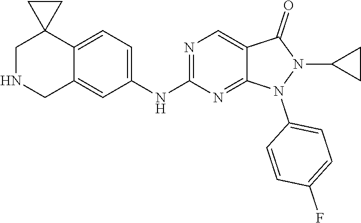 [SMILES: O=c1c2cnc(Nc3ccc4c(c3)CNCC43CC3)nc2n(-c2ccc(F)cc2)n1C1CC1] |
| 1.62 | 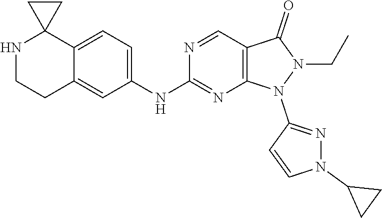 [SMILES: CCn1c(=O)c2cnc(Nc3ccc4c(c3)CCNC43CC3)nc2n1-c1ccn(C2CC2)n1] |
| 1.63 | 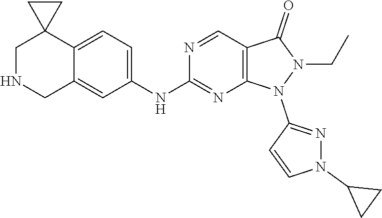 [SMILES: CCn1c(=O)c2cnc(Nc3ccc4c(c3)CNCC43CC3)nc2n1-c1ccn(C2CC2)n1] |
| 1.64 | 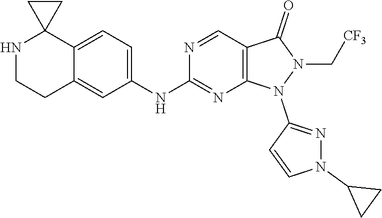 [SMILES: O=c1c2cnc(Nc3ccc4c(c3)CCNC43CC3)nc2n(-c2ccn(C3CC3)n2)n1CC(F)(F)F] |
| 1.65 | 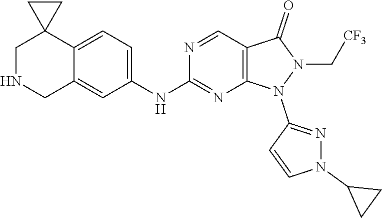 [SMILES: O=c1c2cnc(Nc3ccc4c(c3)CNCC43CC3)nc2n(-c2ccn(C3CC3)n2)n1CC(F)(F)F] |
| 1.66 | 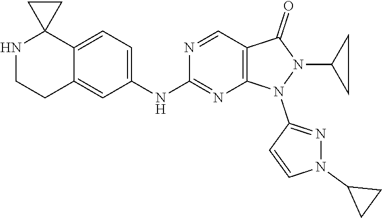 [SMILES: O=c1c2cnc(Nc3ccc4c(c3)CCNC43CC3)nc2n(-c2ccn(C3CC3)n2)n1C1CC1] |
| 1.67 | 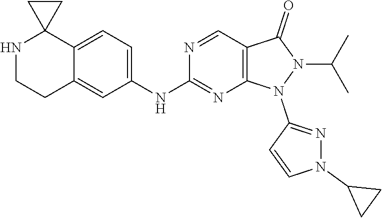 [SMILES: CC(C)n1c(=O)c2cnc(Nc3ccc4c(c3)CCNC43CC3)nc2n1-c1ccn(C2CC2)n1] |
| 1.68 | 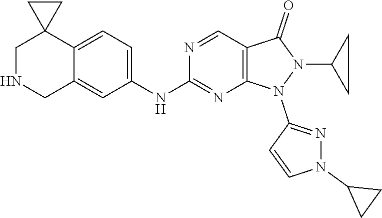 [SMILES: O=c1c2cnc(Nc3ccc4c(c3)CNCC43CC3)nc2n(-c2ccn(C3CC3)n2)n1C1CC1] |
| 1.69 | 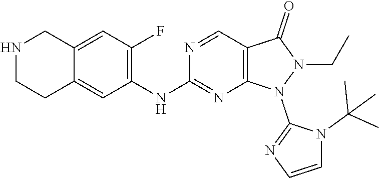 [SMILES: CCn1c(=O)c2cnc(Nc3cc4c(cc3F)CNCC4)nc2n1-c1nccn1C(C)(C)C] |
| 1.70 | 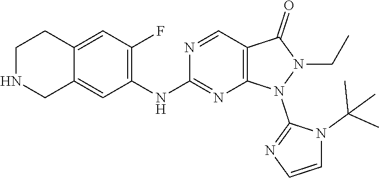 [SMILES: CCn1c(=O)c2cnc(Nc3cc4c(cc3F)CCNC4)nc2n1-c1nccn1C(C)(C)C] |
| 1.71 | 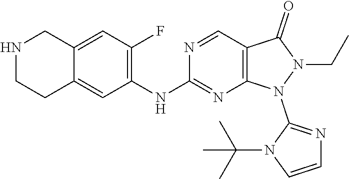 [SMILES: CCn1c(=O)c2cnc(Nc3cc4c(cc3F)CNCC4)nc2n1-c1nccn1C(C)(C)C] |
| 1.72 | 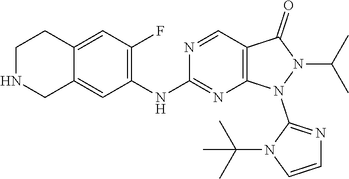 [SMILES: CC(C)n1c(=O)c2cnc(Nc3cc4c(cc3F)CCNC4)nc2n1-c1nccn1C(C)(C)C] |
| 1.73 | 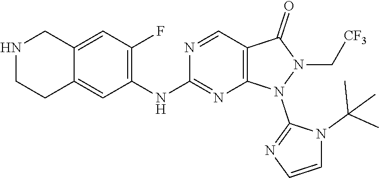 [SMILES: CC(C)(C)n1ccnc1-n1c2nc(Nc3cc4c(cc3F)CNCC4)ncc2c(=O)n1CC(F)(F)F] |
| 1.74 | 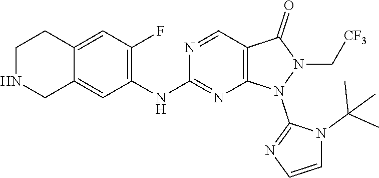 [SMILES: CC(C)(C)n1ccnc1-n1c2nc(Nc3cc4c(cc3F)CCNC4)ncc2c(=O)n1CC(F)(F)F] |
| 1.75 | 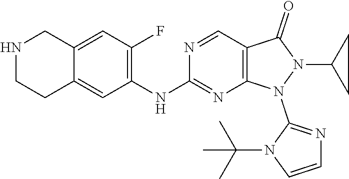 [SMILES: CC(C)(C)n1ccnc1-n1c2nc(Nc3cc4c(cc3F)CNCC4)ncc2c(=O)n1C1CC1] |
| 1.76 | 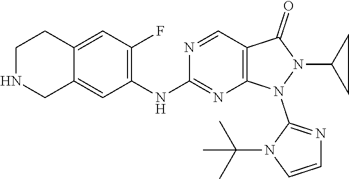 [SMILES: CC(C)(C)n1ccnc1-n1c2nc(Nc3cc4c(cc3F)CCNC4)ncc2c(=O)n1C1CC1] |
| 1.77 | 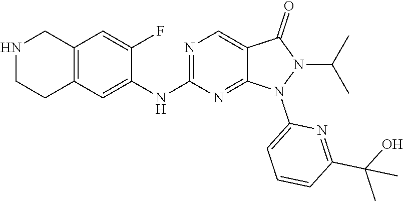 [SMILES: CC(C)n1c(=O)c2cnc(Nc3cc4c(cc3F)CNCC4)nc2n1-c1cccc(C(C)(C)O)n1] |
| 1.78 | 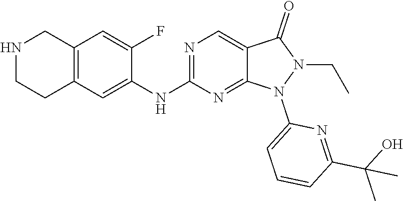 [SMILES: CCn1c(=O)c2cnc(Nc3cc4c(cc3F)CNCC4)nc2n1-c1cccc(C(C)(C)O)n1] |
| 1.79 | 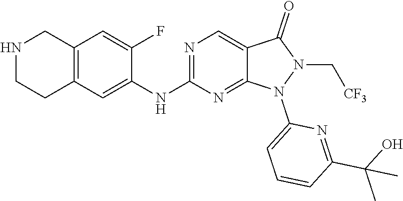 [SMILES: CC(C)(O)c1cccc(-n2c3nc(Nc4cc5c(cc4F)CNCC5)ncc3c(=O)n2CC(F)(F)F)n1] |
| 1.80 | 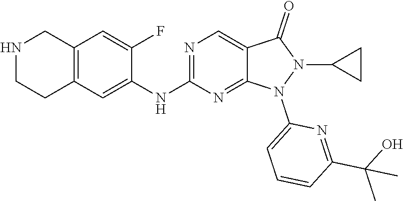 [SMILES: CC(C)(O)c1cccc(-n2c3nc(Nc4cc5c(cc4F)CNCC5)ncc3c(=O)n2C2CC2)n1] |
| 1.81 | 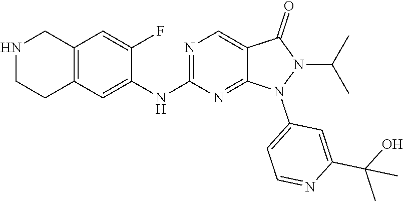 [SMILES: CC(C)n1c(=O)c2cnc(Nc3cc4c(cc3F)CNCC4)nc2n1-c1ccnc(C(C)(C)O)c1] |
| 1.82 | 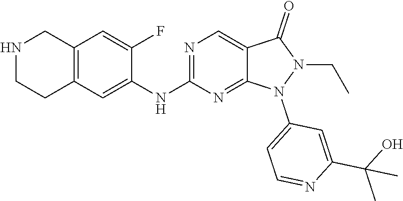 [SMILES: CCn1c(=O)c2cnc(Nc3cc4c(cc3F)CNCC4)nc2n1-c1ccnc(C(C)(C)O)c1] |
| 1.83 | 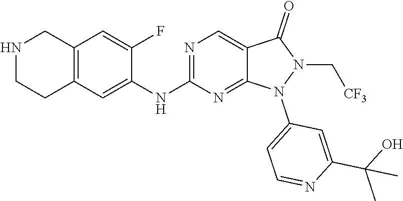 [SMILES: CC(C)(O)c1cc(-n2c3nc(Nc4cc5c(cc4F)CNCC5)ncc3c(=O)n2CC(F)(F)F)ccn1] |
| 1.84 | 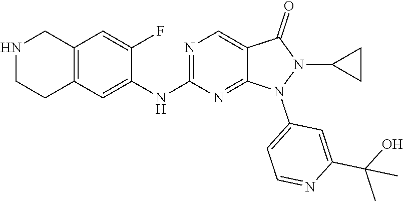 [SMILES: CC(C)(O)c1cc(-n2c3nc(Nc4cc5c(cc4F)CNCC5)ncc3c(=O)n2C2CC2)ccn1] |
| 1.85 | 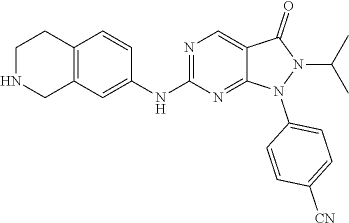 [SMILES: CC(C)n1c(=O)c2cnc(Nc3ccc4c(c3)CNCC4)nc2n1-c1ccc(C#N)cc1] |
| 1.86 | 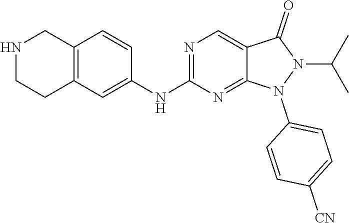 [SMILES: CC(C)n1c(=O)c2cnc(Nc3ccc4c(c3)CCNC4)nc2n1-c1ccc(C#N)cc1] |
| 1.87 | 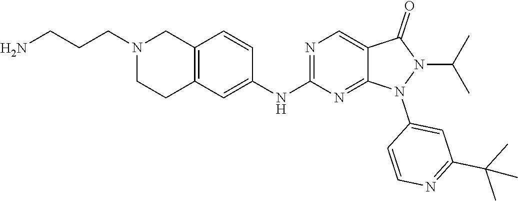 [SMILES: CC(C)n1c(=O)c2cnc(Nc3ccc4c(c3)CCN(CCCN)C4)nc2n1-c1ccnc(C(C)(C)C)c1] |

In some embodiments, provided herein is a compound described in Table 1, or a tautomer thereof, or a salt of any of the foregoing, and uses thereof. In some embodiments, provided herein is a compound described in Table 1 or a pharmaceutically acceptable salt thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described, such as the compounds of Table 1. The structure or name is intended to embrace all possible stereoisomers of a compound depicted, and each unique stereoisomer has a compound number bearing a suffix “a”, “b”, etc. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of Formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Methods

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein or a salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

In some embodiments, compounds of Formula (I), (II) or (III) are synthesized according to Scheme 1 to Scheme 5.

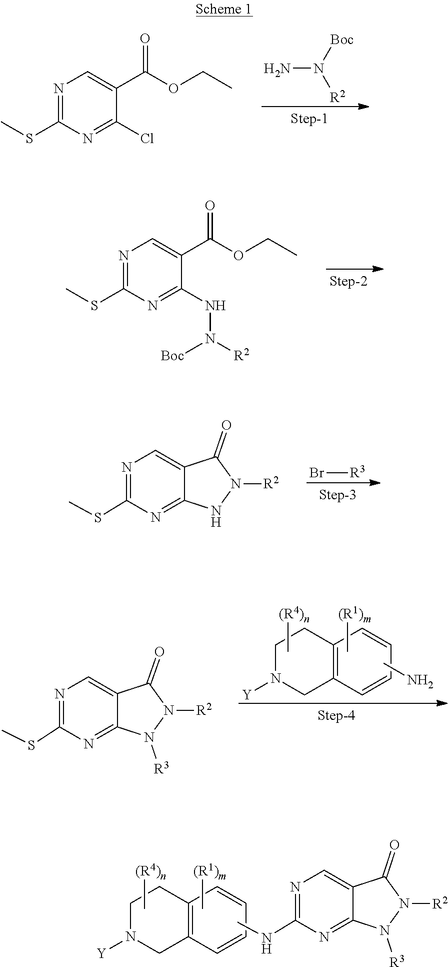

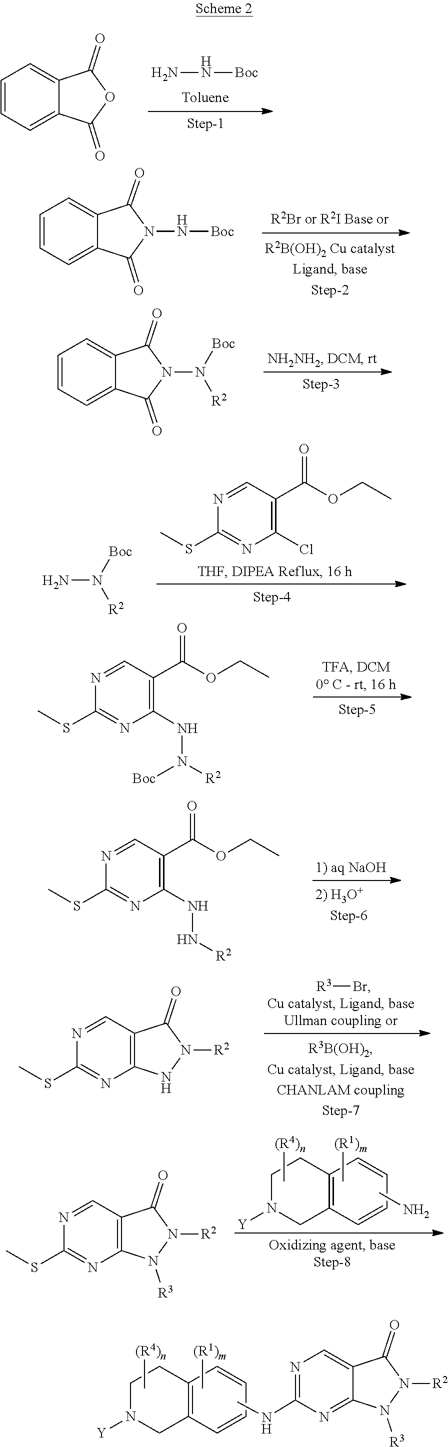

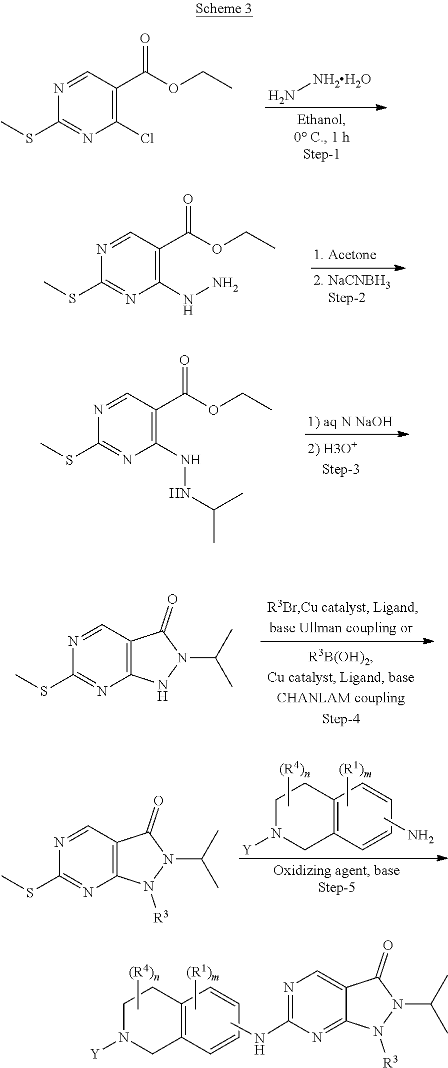

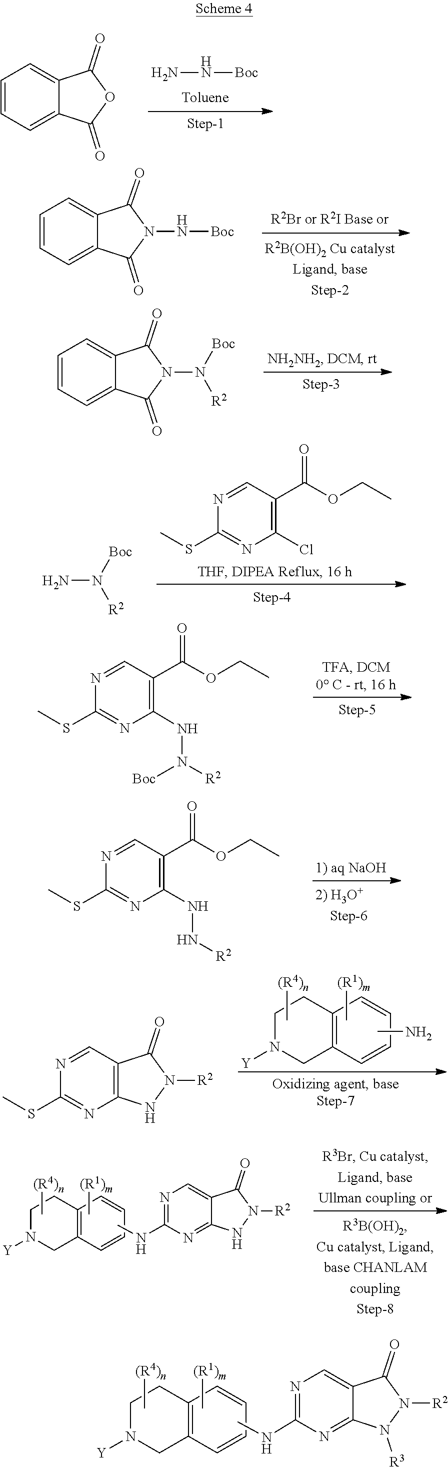

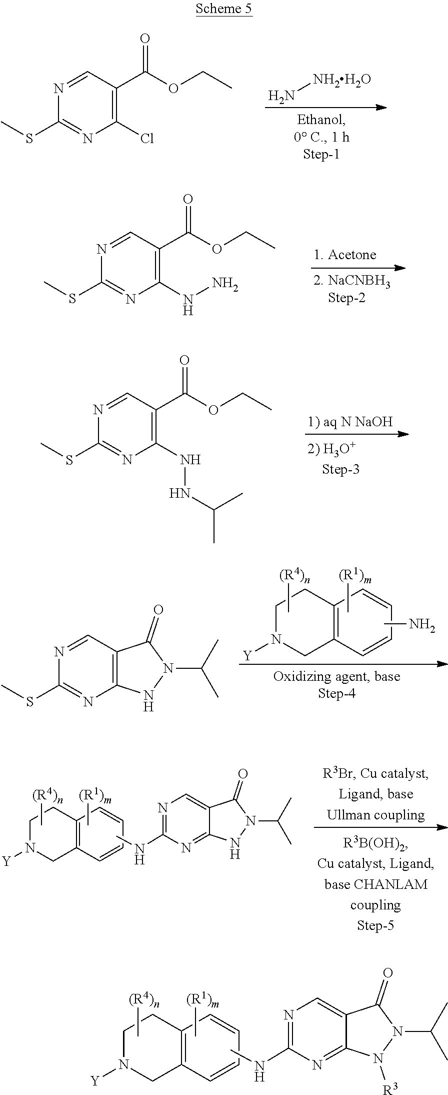

wherein m, n, Y, R¹, R², R³, and R⁴ are as defined herein for Formula (I). Particular examples are provided in the Examples Section below.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

One or several compounds described herein or a salt thereof can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

Compounds as described herein may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease in an individual comprising administering an effective amount of a compound of Formula (I), (II) or (III) or any embodiment, variation or aspect thereof (collectively, a compound of Formula (I), (II) or (III) or the present compounds or the compounds detailed or described herein) or a pharmaceutically acceptable salt thereof, to the individual. Further provided herein is a method of treating a proliferative disease in an individual, comprising administering an effective amount of a compound of Formula (I), (II) or (III) or a pharmaceutically acceptable salt thereof, to the individual. Also provided herein is a method of treating cancer in an individual comprising administering an effective amount of a compound of Formula (I), (II) or (III) or a pharmaceutically acceptable salt thereof, to the individual. In some embodiments, the compound is administered to the individual according to a dosage and/or method of administration described herein.

In some embodiments, the cancer in the individual has one or more TP53 gene mutations or expresses mutant p53. In some embodiments, the cancer in the individual that has one or more TP53 gene mutations or expresses mutant p53 is glioblastoma. TP53 is the human gene that encodes p53. In some embodiments, provided herein is a method of treating a cancer in an individual, comprising (a) selecting the individual for treatment based on (i) the presence of one or more mutations of the TP53 gene in the cancer, or (ii) expression of mutant p53 in the cancer, and administering an effective amount of a compound of Formula (I), (II) or (III) or a pharmaceutically acceptable salt thereof, to the individual. In some embodiments, the cancer is assayed for the expression of mutant p53. In some embodiments, the TP53 gene of the cancer is sequenced to detect the one or more mutations. In some embodiments, the TP53 gene is sequenced by biopsying the cancer and sequencing the TP53 gene from the biopsied cancer. In some embodiments, the TP53 gene is sequenced by sequencing circulating-tumor DNA (ctDNA) from the individual.

In some embodiments, provided herein is a method of using a compound of Formula (I), (II) or (III) or any embodiment in the manufacture of a medicament for treatment of a disease. In some embodiments, provided herein is a method of using a compound of Formula (I), (II) or (III) or any embodiment in the manufacture of a medicament for treatment of cancer.

In some embodiments, a compound of Formula (I), (II) or (III) or a salt thereof is used to treat an individual having a proliferative disease, such as cancer as described herein. In some embodiments, the individual is at risk of developing a proliferative disease, such as cancer. In some of these embodiments, the individual is determined to be at risk of developing cancer based upon one or more risk factors. In some of these embodiments, the risk factor is a family history and/or gene associated with cancer.

The present compounds or salts thereof are believed to be effective for treating a variety of diseases and disorders. For example, in some embodiments, the present compositions may be used to treat a proliferative disease, such as cancer. In some embodiments the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma, cutaneous T cell lymphoma, Hodgkin's lymphoma, non-Hodgkin's lymphoma, cancer of the adrenal cortex, ACTH-producing tumors, lung cancer, including small cell carcinoma and nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, glioblastoma, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the compounds and compositions described herein suppress G₂-M checkpoint in a cell (such as a cancer cell). In some embodiments, the cancer cell is a cancer cell from any of the cancer types described herein. Suppression of the G₂-M DNA damage checkpoint results in premature mitosis of the cell, and consequently apoptosis. In some embodiments, provided herein is a method of suppressing the G₂-M DNA damage checkpoint in a cell comprising administering an effective amount of a compound of Formula (I), (II) or (III) or a pharmaceutically acceptable salt thereof, to the cell. In some embodiments, the G₂-M DNA damage checkpoint is suppressed in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, the G₂-M DNA damage checkpoint is suppressed in up to about 99%, up to about 98%, up to about 97%, up to about 96%, up to about 95%, up to about 90%, up to about 85%, or up to about 80% of cells in the cell population.

In some embodiments, provided herein is a method of inducing premature mitosis in a cell comprising administering an effective amount of a compound of Formula (I), (II) or (III) or a pharmaceutically acceptable salt thereof, to the cell. In some embodiments, premature mitosis is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, premature mitosis is induced in up to about 99%, up to about 98%, up to about 97%, up to about 96%, up to about 95%, up to about 90%, up to about 85%, or up to about 80% of cells in the cell population.

In some embodiments, provided herein is a method of inducing apoptosis in a cell comprising administering an effective amount of a compound of Formula (I), (II) or (III) or a pharmaceutically acceptable salt thereof, to the cell. In some embodiments, apoptosis is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, apoptosis is induced in up to about 99%, up to about 98%, up to about 97%, up to about 96%, up to about 95%, up to about 90%, up to about 85%, or up to about 80% of cells in the cell population.

In some embodiments, provided herein is a method of inhibiting Wee1 in a cell comprising administering an effective amount of a compound of Formula (I), (II) or (III) or a pharmaceutically acceptable salt thereof, to the cell. In some embodiments, Wee1 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, Wee1 is inhibited up to about 99%, up to about 98%, up to about 97%, up to about 96%, up to about 95%, up to about 90%, up to about 85%, up to about 80%, up to about 70%, or up to about 60%. In some embodiments, the activity of Wee1 is measured according to a kinase assay.

In some embodiments, provided herein is a method of inhibiting Wee1 comprising contacting Wee1 with an effective amount of a compound of Formula (I), (II) or (III) or a pharmaceutically acceptable salt thereof. In some embodiments, a compound of Formula (I), (II) or (III) or a pharmaceutically acceptable salt thereof binds to Wee1 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, a compound of Formula (I), (II) or (III) or a pharmaceutically acceptable salt thereof binds to Wee1 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell cytotoxicity assay.

In some embodiments, provided herein is a method of inhibiting the proliferation of a cell, comprising contacting the cell with an effective amount of a compound of Formula (I), (II) or (III) or a pharmaceutically acceptable salt thereof. In some embodiments, a compound of Formula (I), (II) or (III) or a pharmaceutically acceptable salt thereof is effective in inhibiting the proliferation of the cell with an IC₅₀ of less than 5 μM, less than 2 μM, less than 1 μM, less than 900 nM, less than 800 nM, less than 700 nM, less than 600 nM, less than 500 nM, less than 400 nM, less than 300 nM, less than 200 nM, less than 100 nM, or less than 50 nM. In some embodiments, a compound of Formula (I), (II) or (III) or a pharmaceutically acceptable salt is effective in inhibiting the proliferation of the cell with an IC₅₀ between 10 nM and 20 nM, between 20 nM and 50 nM, between 50 nM and 100 nM, between 100 nM and 500 nM, between 500 nM and 1 μM, between 1 μM and 2 μM, or between 2 μM and 5 μM. In some embodiments, the IC₅₀ is measured according to a cell proliferation assay.

### Combination Therapy

As provided herein, the presently disclosed compounds or a salt thereof may activate the immune system, for example by inducing apoptosis or suppressing mitosis of cancer cells. Accordingly, the present compounds or a salt thereof may be used in combination with other anti-cancer agents to enhance tumor immunotherapy. In some embodiments, provided herein is a method of treating a disease in an individual comprising administering an effective amount of a compound of Formula (I), (II) or (III) or any embodiment, variation or aspect thereof (collectively, a compound of Formula (I), (II) or (III) or the present compounds or the compounds detailed or described herein) or a pharmaceutically acceptable salt thereof, and an additional therapeutic agent to the individual. In some embodiments, the disease is a proliferative disease such as cancer.

In some embodiments, the additional therapeutic agent is a cancer immunotherapy agent. In some embodiments, the additional therapeutic agent is a chemotherapeutic agent. In some embodiments, the additional therapeutic agent is an immunostimulatory agent. In some embodiments, the additional therapeutic agent targets a checkpoint protein (for example an immune checkpoint inhibitor). In some embodiments, the additional therapeutic agent is effective to stimulate, enhance or improve an immune response against a tumor. In some embodiments, the additional chemotherapeutic agent is a DNA alkylating agent, a platinum-based chemotherapeutic agent, a kinase inhibitor or a DNA damage repair (DDR) pathway inhibitor. In some embodiments, the additional chemotherapeutic agent is a DNA alkylating agent. In some embodiments, the additional chemotherapeutic agent is a platinum-based chemotherapeutic agent. In some embodiments, the additional chemotherapeutic agent is a kinase inhibitor. In some embodiments, the additional chemotherapeutic agent is a DNA damage repair (DDR) pathway inhibitor.

In another aspect, provided herein is a combination therapy for the treatment of a disease, such as cancer. In some embodiments, provided herein is a method of treating a disease in an individual comprising administering an effective amount of a compound of Formula (I), (II) or (III) or any embodiment, variation or aspect thereof (collectively, a compound of Formula (I), (II) or (III) or the present compounds or the compounds detailed or described herein) or a pharmaceutically acceptable salt thereof, in combination with a radiation therapy.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of an additional chemotherapeutic agent. In some embodiments, the chemotherapeutic agent is a kinase inhibitor or an agent that inhibits one or more DNA damage repair (DDR) pathways. In some embodiments, a compound of Formula (I), (II) or (III) or a pharmaceutically acceptable salt thereof is administered prior to, after, or simultaneously co-administered with the additional chemotherapeutic agent. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the additional chemotherapeutic agent.

Examples of chemotherapeutic agents that can be used in combination with a compound of Formula (I), (II) or (III) or a pharmaceutically acceptable salt thereof include DNA-targeted agents, a DNA alkylating agent (such as cyclophosphamide, mechlorethamine, chlorambucil, melphalan, dacarbazine, temozolomide or nitrosoureas), a topoisomerase inhibitor (such as a Topoisomerase I inhibitor (e.g., irinotecan or topotecan) or a Topoisomerase II inhibitor (e.g., etoposide or teniposide)), an anthracycline (such as daunorubicin, doxorubicin, epirubicin, idarubicin, mitoxantrone, or valrubicin), a histone deacetylase inhibitor (such as vorinostat or romidepsin), a bromodomain inhibitor, other epigenetic inhibitors, a taxane (such as paclitaxel or docetaxel), a kinase inhibitor (such as bortezomib, erlotinib, gefitinib, imatinib, vemurafenib, or vismodegib), an anti-angiogenic inhibitor, a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine), or a platinum-based chemotherapeutic agent (such as cisplatin, carboplatin, or oxaliplatin), pemetrexed, or a combination thereof. In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a kinase inhibitor (such as bortezomib, erlotinib, gefitinib, imatinib, vemurafenib, or vismodegib). In some embodiments, a compound of Formula (I), (II) or (III) or a pharmaceutically acceptable salt thereof is administered prior to, after, or simultaneously co-administered with the kinase inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the kinase inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a DNA damaging agent. In some embodiments, a compound of Formula (I), (II) or (III) or a pharmaceutically acceptable salt thereof is administered prior to, after, or simultaneously co-administered with the DNA damaging agent. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA damaging agent.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a DNA alkylating agent (such as cyclophosphamide, mechlorethamine, chlorambucil, melphalan, dacarbazine, temozolomide or nitrosoureas). In some embodiments, a compound of Formula (I), (II) or (III) or a pharmaceutically acceptable salt thereof is administered prior to, after, or simultaneously co-administered with the DNA alkylating agent. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the DNA alkylating agent.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a topoisomerase inhibitor (such as a Topoisomerase I inhibitor (e.g., irinotecan or topotecan) or a Topoisomerase II inhibitor (e.g., etoposide or teniposide)). In some embodiments, a compound of Formula (I), (II) or (III) or a pharmaceutically acceptable salt thereof is administered prior to, after, or simultaneously co-administered with the topoisomerase inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the topoisomerase inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of an anthracycline (such as daunorubicin, doxorubicin, epirubicin, idarubicin, mitoxantrone, or valrubicin). In some embodiments, a compound of Formula (I), (II) or (III) or a pharmaceutically acceptable salt thereof is administered prior to, after, or simultaneously co-administered with the anthracycline. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the anthracycline.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a histone deacetylase inhibitor (such as vorinostat or romidepsin). In some embodiments, a compound of Formula (I), (II) or (III) or a pharmaceutically acceptable salt thereof is administered prior to, after, or simultaneously co-administered with the histone deacetylase inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the histone deacetylase inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a taxane (such as paclitaxel or docetaxel). In some embodiments, a compound of Formula (I), (II) or (III) or a pharmaceutically acceptable salt thereof is administered prior to, after, or simultaneously co-administered with the taxane. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the taxane.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine). In some embodiments, a compound of Formula (I), (II) or (III) or a pharmaceutically acceptable salt thereof is administered prior to, after, or simultaneously co-administered with the nucleotide analog or precursor analog. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the nucleotide analog or precursor analog.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a platinum-based chemotherapeutic agent (such as cisplatin, carboplatin, or oxaliplatin). In some embodiments, a compound of Formula (I), (II) or (III) or a pharmaceutically acceptable salt thereof is administered prior to, after, or simultaneously co-administered with the platinum-based chemotherapeutic agent. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the platinum-based chemotherapeutic agent.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of pemetrexed. In some embodiments, a compound of Formula (I), (II) or (III) or a pharmaceutically acceptable salt thereof is administered prior to, after, or simultaneously co-administered with the pemetrexed. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the pemetrexed.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a DDR pathway inhibitor. In some embodiments, a compound of Formula (I), (II) or (III) or a pharmaceutically acceptable salt thereof is administered prior to, after, or simultaneously co-administered with the DDR pathway inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the DDR pathway inhibitor. Examples of inhibitors of the DDR pathway include poly(ADP-ribose) polymerase (PARP) inhibitors (such as olaparib, rucaparib, niraparib, or talazoparib), ataxia telangiectasia mutated (ATM) protein inhibitors, ataxia telangiectasia and Rad3-related (ATR) protein inhibitors, checkpoint kinase 1 (Chk1) inhibitors, or combinations thereof.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of a PARP inhibitor (such as olaparib, rucaparib, niraparib, or talazoparib). In some embodiments, a compound of Formula (I), (II) or (III) or a pharmaceutically acceptable salt thereof is administered prior to, after, or simultaneously co-administered with the PARP inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the PARP inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of an ATM protein inhibitor. In some embodiments, a compound of Formula (I), (II) or (III) or a pharmaceutically acceptable salt thereof is administered prior to, after, or simultaneously co-administered with the ATM protein inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the ATM protein inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of an ATR protein inhibitor. In some embodiments, a compound of Formula (I), (II) or (III) or a pharmaceutically acceptable salt thereof is administered prior to, after, or simultaneously co-administered with the ATR protein inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the ATR protein inhibitor.

In some embodiments, provided herein is a method of treating a disease in an individual comprising (a) administering an effective amount of a compound of Formula (I), (II) or (III) or any embodiment, variation or aspect thereof (collectively, Formula (I), (II) or (III)) or a pharmaceutically acceptable salt thereof, and (b) administering an effective amount of an Chk1 inhibitor. In some embodiments, a compound of Formula (I), (II) or (III) or a pharmaceutically acceptable salt thereof is administered prior to, after, or simultaneously co-administered with the Chk1 inhibitor. In some embodiments, a compound of Formula (I), (II) or (III) or a pharmaceutically acceptable salt thereof is administered 1 or more hours (such as 2 or more hours, 4 or more hours, 8 or more hours, 12 or more hours, 24 or more hours, or 48 or more hours) prior to or after the Chk1 inhibitor.

In another aspect, provided herein is a combination therapy in which a compound of Formula (I), (II) or (III) or a salt thereof is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a compound of Formula (I), (II) or (III) or a salt thereof and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a compound of Formula (I), (II) or (III) or a salt thereof and an anti-PD-1 antibody. In another embodiment, the subject is administered a compound of Formula (I), (II) or (III) or a salt thereof and an anti-PD-L1 antibody. In yet another embodiment, the subject is administered a compound of formula (I) or a salt thereof and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody).

In one embodiment, the present disclosure provides a method for treating a proliferative disease (e.g., cancer), comprising administering a compound of Formula (I), (II) or (III) or a salt thereof and an anti-PD-1 antibody to a subject. In further embodiments, a compound of Formula (I), (II) or (III) or a salt thereof is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a compound of Formula (I), (II) or (III) or a salt thereof and a subtherapeutic dose of anti-PD-1 antibody to a subject. In certain embodiments, the subject is human. In certain embodiments, the anti-PD-1 antibody is a human sequence monoclonal antibody.

In one embodiment, the present invention provides a method for treating a hyperproliferative disease (e.g., cancer), comprising administering a compound of Formula (I), (II) or (III) or a salt thereof and an anti-PD-L1 antibody to a subject. In further embodiments, a compound of Formula (I), (II) or (III) or a salt thereof is administered at a subtherapeutic dose, the anti-PD-L1 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering a compound of Formula (I), (II) or (III) or a salt thereof and a subtherapeutic dose of anti-PD-L1 antibody to a subject. In certain embodiments, the subject is human. In certain embodiments, the anti-PD-L1 antibody is a human sequence monoclonal antibody.

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a compound of Formula (I), (II) or (III) or a salt thereof can be administered sequentially, such as anti-CTLA-4 antibody being administered first and a compound of Formula (I), (II) or (III) or a salt thereof second, or a compound of formula Formula (I), (II) or (III) or a salt thereof being administered first and anti-CTLA-4 antibody second. Additionally or alternatively, an anti-PD-1 antibody and a compound of Formula (I), (II) or (III) or a salt thereof can be administered sequentially, such as anti-PD-1 antibody being administered first and a compound of Formula (I), (II) or (III) or a salt thereof second, or a compound of Formula (I), (II) or (III) or a salt thereof being administered first and anti-PD-1 antibody second. Additionally or alternatively, an anti-PD-L1 antibody and a compound of Formula (I), (II) or (III) or a salt thereof can be administered sequentially, such as anti-PD-L1 antibody being administered first and a compound of Formula (I), (II) or (III) or a salt thereof second, or a compound of Formula (I), (II) or (III) or a salt thereof being administered first and anti-PD-L1 antibody second.

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination of a compound of Formula (I), (II) or (III) or a salt thereof can be further combined with an immunogenic agent, such as cancerous cells, purified tumor antigens (including recombinant proteins, peptides, and carbohydrate molecules), cells, and cells transfected with genes encoding immune stimulating cytokines.

A compound of Formula (I), (II) or (III) or a salt thereof can also be further combined with standard cancer treatments. For example, a compound of Formula (I), (II) or (III) or a salt thereof can be effectively combined with chemotherapeutic regimes. In these instances, it is possible to reduce the dose of other chemotherapeutic reagent administered with the combination of the instant disclosure (Mokyr et al. (1998) Cancer Research 58: 5301-5304). Other combination therapies with a compound of Formula (I), (II) or (III) or a salt thereof include radiation, surgery, or hormone deprivation. Angiogenesis inhibitors can also be combined with a compound of Formula (I), (II) or (III) or a salt thereof. Inhibition of angiogenesis leads to tumor cell death, which can be a source of tumor antigen fed into host antigen presentation pathways.

In another example, a compound of Formula (I), (II) or (III) or a salt thereof can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or a compound of Formula (I), (II) or (III) or a salt thereof. In an exemplary embodiment, a treatment of a hyperproliferative disease (e.g., a cancer tumor) can include an anti-cancer antibody in combination with a compound of Formula (I), (II) or (III) or a salt thereof and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a compound of Formula (I), (II) or (III) or a salt thereof.

In some embodiments, a compound of Formula (I), (II) or (III) or a salt thereof can be combined with an anti-CD73 therapy, such as an anti-CD73 antibody.

In yet further embodiments, a compound of Formula (I), (II) or (III) or a salt thereof is administered in combination with another Wee1 inhibitor.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the invention may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition of the invention may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14 day time period, such as about 2 months, about 4 months, about 6 months or more). Any of the dosing frequencies can employ any of the compounds described herein together with any of the dosages described herein.

The compounds provided herein or a salt thereof may be administered to an individual via various routes, including, e.g., intravenous, intramuscular, subcutaneous, oral and transdermal. A compound provided herein can be administered frequently at low doses, known as ‘metronomic therapy,’ or as part of a maintenance therapy using compound alone or in combination with one or more additional drugs. Metronomic therapy or maintenance therapy can comprise administration of a compound provided herein in cycles. Metronomic therapy or maintenance therapy can comprise intra-tumoral administration of a compound provided herein.

In one aspect, the invention provides a method of treating cancer in an individual by parenterally administering to the individual (e.g., a human) an effective amount of a compound or salt thereof. In some embodiments, the route of administration is intravenous, intra-arterial, intramuscular, or subcutaneous. In some embodiments, the route of administration is oral. In still other embodiments, the route of administration is transdermal.

The invention also provides compositions (including pharmaceutical compositions) as described herein for the use in treating, preventing, and/or delaying the onset and/or development of cancer and other methods described herein. In certain embodiments, the composition comprises a pharmaceutical formulation which is present in a unit dosage form.

Also provided are articles of manufacture comprising a compound of the disclosure or a salt thereof, composition, and unit dosages described herein in suitable packaging for use in the methods described herein. Suitable packaging is known in the art and includes, for example, vials, vessels, ampules, bottles, jars, flexible packaging and the like. An article of manufacture may further be sterilized and/or sealed.

### Kits

The present disclosure further provides kits for carrying out the methods of the invention, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

### Synthetic Examples

### Example S-1: Synthesis of 2-isopropyl-1-(1-isopropyl-1H-pyrazol-4-yl)-6-((1,2,3,4-tetrahydroisoquinolin-6-yl)amino)-1,2-dihydro-3H-pyrazolo[3,4-d]pyrimidin-3-one (Compound No. 1.1)

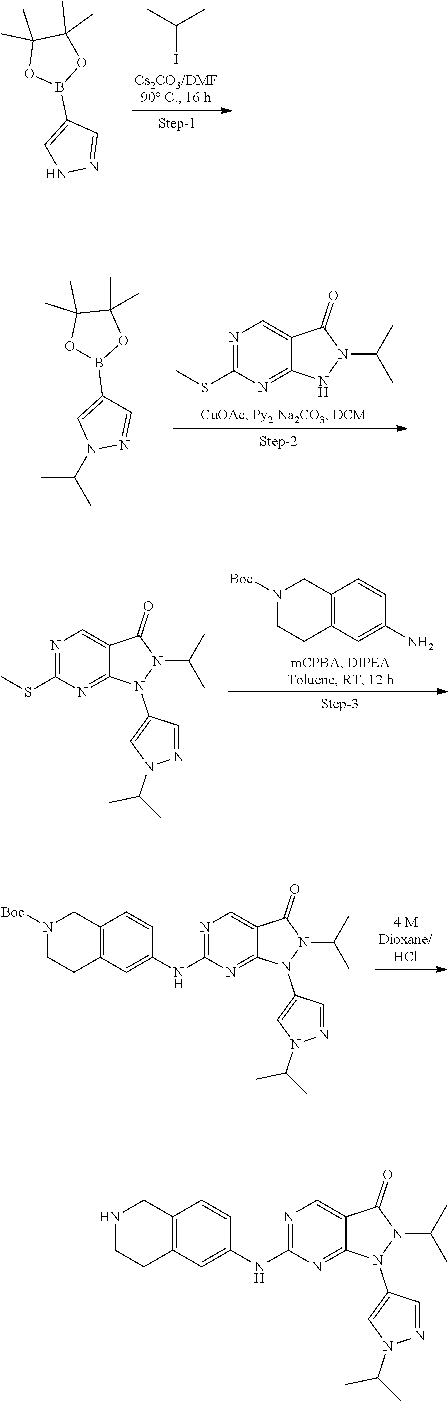

Step-1: Synthesis of 1-isopropyl-4-(4,4,5,5-tetramethyl-1,3,2-dioxaborolan-2-yl)-1H-pyrazole: To a solution of 4-(4,4,5,5-tetramethyl-1,3,2-dioxaborolan-2-yl)-1H-pyrazole (2.0 g, 10.30 mmol, 1.0 eq) in DMF (30 mL) was added 2-iodopropane (2.45 mL, 15.46 mmol, 1.5 eq) and Cs₂CO₃ (5.36 g, 16.48 mmol). The reaction mixture was heated at 90° C. for 16 h. After completion of reaction, the reaction mixture was diluted with water and extracted with EtOAc (250 mL×2). The combined organic layers were washed with water (250 mL) and brine solution (250 mL), dried over anhydrous sodium sulfate and concentrated under reduced pressure to afford crude product, which was purified by flash chromatography [silica gel 100-200 mesh; elution 0-40% EtOAc in hexane] to afford the desired compound (1.5 g, 61.72%) as brown solid. LCMS: 237.2 [M+1]⁺.

Step-2: Synthesis of 2-isopropyl-1-(1-isopropyl-1H-pyrazol-4-yl)-6-(methylthio)-1,2-dihydro-3H-pyrazolo[3,4-d]pyrimidin-3-one: To a stirred solution of 2-isopropyl-6-(methylthio)-1,2-dihydro-3H-pyrazolo[3,4-d]pyrimidin-3-one (861 mg, 3.84 mmol, 1 eq) and 1-isopropyl-4-(4,4,5,5-tetramethyl-1,3,2-dioxaborolan-2-yl)-1H-pyrazole (1.0 g, 4.23 mmol, 1.1 eq) in DCM (30 mL) was added 2,2-bipyridine (899 mg, 5.76 mmol, 1.5 eq), copper acetate (1.04 g, 5.76 mmol, 1.5 eq) and Na₂CO₃ (1.22 g, 11.52 mmol, 3.0 eq). The reaction mixture was stirred at RT for 24 h in open air. The progress of reaction was monitored by TLC. After completion, the reaction mixture was filtered over celite to remove inorganic impurities. The filtrate was washed with water, dried over Na₂SO₄, concentrated and purified by column chromatography (Combiflash, Elution: 0-30% EtOAc in Hexane) to afford the desired product (110 mg, 8.6%) as an off-white solid. LCMS: 333.2 [M+1]⁺.

Step-3: Synthesis of tert-butyl 6-((2-isopropyl-1-(1-isopropyl-1H-pyrazol-4-yl)-3-oxo-2,3-dihydro-1H-pyrazolo[3,4-d]pyrimidin-6-yl)amino)-3,4-dihydroisoquinoline-2(1H)-carboxylate: To a stirring solution of 2-isopropyl-1-(1-isopropyl-1H-pyrazol-4-yl)-6-(methylthio)-1,2-dihydro-3H-pyrazolo[3,4-d]pyrimidin-3-one (110 mg, 0.33 mmol, 1.0 eq) in toluene (3.0 mL) was added mCPBA (114 mg, 0.66 mmol, 2.0 eq) and allowed to stir at RT for 1 h. Tert-butyl 6-amino-3,4-dihydroisoquinoline-2(1H)-carboxylate (98 mg, 0.39 mmol, 1.0 eq) and DIPEA (0.20 mL, 1.32 mmol, 4.0 eq) were added and allowed to stir at RT overnight. After completion of the reaction, the reaction mixture was diluted with water and extracted with EtOAc (50 mL×2). The combined organic layers were washed with water (50 mL) and brine solution (50 mL), dried over anhydrous sodium sulfate and concentrated under reduced pressure to afford crude product, which was purified by flash chromatography [silica gel 100-200 mesh; elution 0-70% EtOAc in hexane] to afford the desired compound (70 mg, 39.7%) as light brown solid. LCMS: 533.3 [M+1]⁺.

Step-4: Synthesis of 2-isopropyl-1-(1-isopropyl-1H-pyrazol-4-yl)-6-((1,2,3,4-tetrahydroisoquinolin-6-yl)amino)-1,2-dihydro-3H-pyrazolo[3,4-d]pyrimidin-3-one: Tert-butyl 6-((2-isopropyl-1-(1-isopropyl-1H-pyrazol-4-yl)-3-oxo-2,3-dihydro-1H-pyrazolo[3,4-d]pyrimidin-6-yl)amino)-3,4-dihydroisoquinoline-2(1H)-carboxylate (70 mg, 0.13 mmol, 1.0 eq) was dissolved in dioxane (1 mL), followed by dropwise addition of 4.0 M-HCl in dioxane (1 mL) and allowed to stir at RT for 1 h. After completion of the reaction, the reaction mixture was dried under reduced pressure and purified by reverse phase HPLC to afford the desired compound (30 mg, 52.7%) as light yellow solid. LCMS: 433.3 [M+1]⁺; ¹H NMR (400 MHz, DMSO-d₆, Free base): δ 10.13 (br s, 1H) 8.78 (s, 1H) 8.26 (s, 1H) 7.71 (s, 1H) 7.60 (br s, 1H) 7.47 (d, J=8.3 Hz, 1H) 7.03 (d, J=8.3 Hz, 1H) 4.50-4.60 (m, 1H) 4.12-4.22 (m, 1H) 4.08 (s, 2H) 3.24 (br s, 2H) 2.81 (br s, 2H) 1.46 (d, J=7.0 Hz, 6H) 1.16-1.35 (m, 6H).

### Example S-2: Synthesis of 2-isopropyl-1-(6-(1-methoxy-2-methylpropan-2-yl)pyridin-2-yl)-6-((1,2,3,4-tetrahydroisoquinolin-7-yl)amino)-1,2-dihydro-3H-pyrazolo[3,4-d]pyrimidin-3-one (Compound No. 1.2)

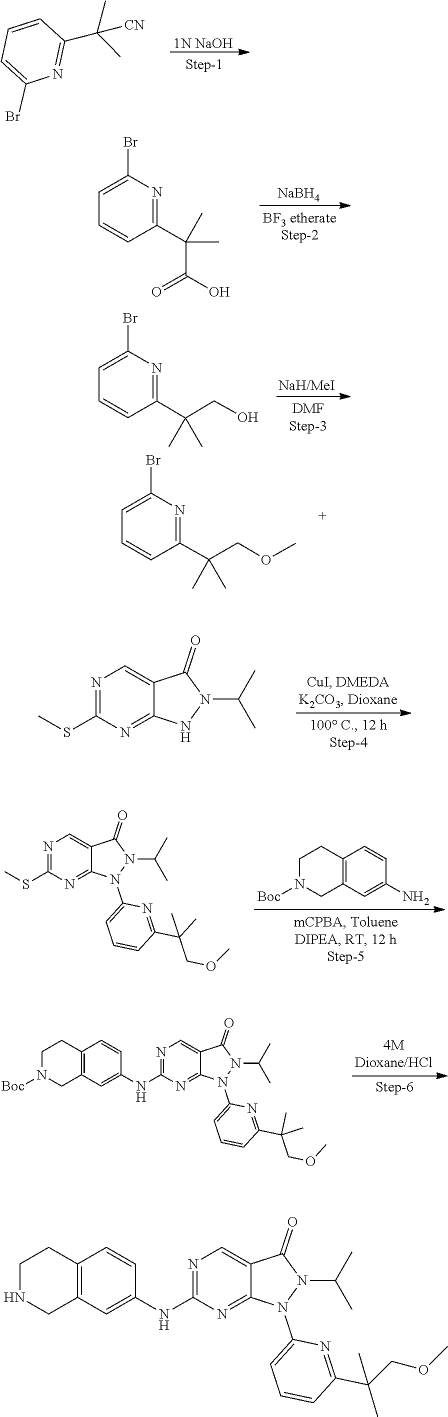

Step-1: Synthesis of 2-(6-bromopyridin-2-yl)-2-methylpropanoic acid: To a solution of 2-(6-bromopyridin-2-yl)-2-methylpropanenitrile (1.5 g, 6.17 mmol, 1.0 eq) in methanol (20 mL) was added 1 N NaOH (20 mL) and the reaction mixture was heated at 100° C. overnight. Progress of the reaction was monitored by TLC analysis. After completion of the reaction, solution was acidified with 2M HCl and the product was extracted into ethyl acetate. Organic layer was dried over sodium sulfate and concentrated to give the desired product (1.2 g, 80.0%) as white solid. LCMS: 243.9 [M+1]⁺.

Step-2: Synthesis of 2-(6-bromopyridin-2-yl)-2-methylpropan-1-ol: To a solution of 2-(6-bromopyridin-2-yl)-2-methylpropanoic acid (1.22 g, 5.0 mmol, 1.0 eq) in dry THF (20 mL) was added sodium borohydride (370 mg, 10 mmol, 2.0 eq), followed by dropwise addition of BF₃.Et₂O (3.0 mL, 20.0 mmol, 1.0 eq). The reaction mixture was then refluxed at 70° C. and the progress of the reaction was monitored by TLC analysis. After completion of the reaction, aqueous ammonium chloride was added and the product was extracted into ethyl acetate. The organic layers were dried over sodium sulfate and concentrated to give the crude product, which was purified by flash chromatography [silica gel 100-200 mesh; elution 0-20% EtOAc in hexane] to afford the desired compound as viscous liquid. LCMS: 229.9 [M+1]⁺.

Step-3: Synthesis of 2-bromo-6-(1-methoxy-2-methylpropan-2-yl)pyridine: To a solution of 2-(6-bromopyridin-2-yl)-2-methylpropan-1-ol (456 mg, 2.0 mmol, 1.0 eq) in DMF at 0° C. was added NaH (160 mg, 4.0 mmol, 2.0 eq) and stirred at RT for 30 minutes. To this solution was added Mel (0.25 mL, 4.0 mmol, 2.0 eq) and stirred for 2 h. Progress of the reaction was monitored by TLC analysis. After completion of the reaction, aqueous ammonium chloride was added and the product was extracted in to ethyl acetate (100 mL×3). The organic layer was dried over sodium sulfate and concentrated to give the crude product, which was purified by flash chromatography [silica gel 100-200 mesh; elution 0-20% EtOAc in hexane] to afford the desired compound (300 mg, 61.4%) as viscous liquid. LCMS: 243.9 [M+1]⁺.

Step-4: Synthesis of 2-isopropyl-1-(6-(1-methoxy-2-methylpropan-2-yl)pyridin-2-yl)-6-(methylthio)-1,2-dihydro-3H-pyrazolo[3,4-d]pyrimidin-3-one: To a stirred solution of 2-isopropyl-6-(methylthio)-1,2-dihydro-3H-pyrazolo[3,4-d]pyrimidin-3-one (224 mg, 1.0 mmol, 1.0 eq) and 2-bromo-6-(1-methoxy-2-methylpropan-2-yl)pyridine (242 mg, 1.0 mmol, 1.0 eq) in (10 mL) of dioxane were added potassium carbonate (276 mg, 2.0 mmol, 2.0 eq) and the resulting mixture was purged with nitrogen for 10 min; followed by addition of copper iodide (38 mg, 0.2 mmol, 0.2 eq), and N,N′-dimethylethylenediamine (DMEDA) (0.05 mL, 0.4 mmol, 0.4 eq) and again purged with nitrogen for 10 min, stirred at 90° C. for overnight. After completion of reaction, the reaction mixture was diluted with water (50 mL) and extracted with EtOAc (50 mL×2). The combined organic layers were washed with water (50 mL) and brine solution (50 mL), dried over anhydrous sodium sulphate and concentrated under reduced pressure to afford crude product, which was purified by flash chromatography to afford the desired compound (300 mg, 77.5%) as an off-white solid. LCMS: 388.3 [M+1]⁺.

Step-5: Synthesis of tert-butyl 7-((2-isopropyl-1-(6-(1-methoxy-2-methylpropan-2-yl)pyridin-2-yl)-3-oxo-2,3-dihydro-1H-pyrazolo[3,4-d]pyrimidin-6-yl)amino)-3,4-dihydroisoquinoline-2(1H)-carboxylate: To a stirred solution of 2-isopropyl-1-(6-(1-methoxy-2-methylpropan-2-yl)pyridin-2-yl)-6-(methylthio)-1,2-dihydro-3H-pyrazolo[3,4-d]pyrimidin-3-one (193 mg, 0.5 mmol, 1.0 eq) in toluene (2.0 mL) was added mCPBA (245 mg, 1.0 mmol, 2.0 eq) and allowed to stir at RT for 1 h. Tert-butyl 6-amino-3,4-dihydroisoquinoline-2(1H)-carboxylate (149 mg, 0.6 mmol, 1.2 eq) and DIPEA (0.44 mL, 2.5 mmol, 5.0 eq) were added and allowed to stir at RT for overnight. After completion of reaction, the reaction mixture was diluted with water and extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (45 mg, 14.8%) as brown solid. LCMS: 588.4 [M+1]⁺.

Step-6: Synthesis of 2-isopropyl-1-(6-(1-methoxy-2-methylpropan-2-yl)pyridin-2-yl)-6-((1,2,3,4-tetrahydroisoquinolin-7-yl)amino)-1,2-dihydro-3H-pyrazolo[3,4-d]pyrimidin-3-one: Tert-butyl 7-((2-isopropyl-1-(6-(1-methoxy-2-methylpropan-2-yl)pyridin-2-yl)-3-oxo-2,3-dihydro-1H-pyrazolo[3,4-d]pyrimidin-6-yl)amino)-3,4-dihydroisoquinoline-2(1H)-carboxylate (40 mg, 0.06 mmol, 1.0 eq) was dissolved in dioxane (1.0 mL), followed by dropwise addition of 4.0 M-HCl in dioxane (1.0 mL) and allowed to stir at RT for 1 h. After completion of the reaction, the solvent was evaporated to give the crude product, which was purified reverse phase chromatography to afford the desired compound, 2-isopropyl-1-(6-(1-methoxy-2-methylpropan-2-yl)pyridin-2-yl)-6-((1,2,3,4-tetrahydroisoquinolin-7-yl)amino)-1,2-dihydro-3H-pyrazolo[3,4-d]pyrimidin-3-one (10 mg, 30.3%) as an off-white solid as formate salt. LCMS: 488.3 [M+1]⁺; ¹H NMR (400 MHz, DMSO-d₆, Formate salt): δ 10.21 (br s, 1H) 8.81 (s, 1H) 8.23 (s, 1H) 8.00 (t, J=7.9 Hz, 1H) 7.75 (d, J=7.9 Hz, 1H) 7.59 (br s, 1H) 7.40 (d, J=7.9 Hz, 2H) 7.06 (d, J=8.7 Hz, 1H) 4.14-4.21 (m, 1H) 3.99 (s, 2H) 3.50 (s, 2H) 3.07-3.19 (m, 5H) 2.76 (br s, 2H) 1.38 (d, J=7.0 Hz, 6H) 1.29 (s, 6H).

### Example S-3: Synthesis of 3-(2-isopropyl-3-oxo-6-((1,2,3,4-tetrahydroisoquinolin-6-yl)amino)-2,3-dihydro-1H-pyrazolo[3,4-d]pyrimidin-1-yl)-N,N-dimethylbenzamide (Compound No. 1.3)

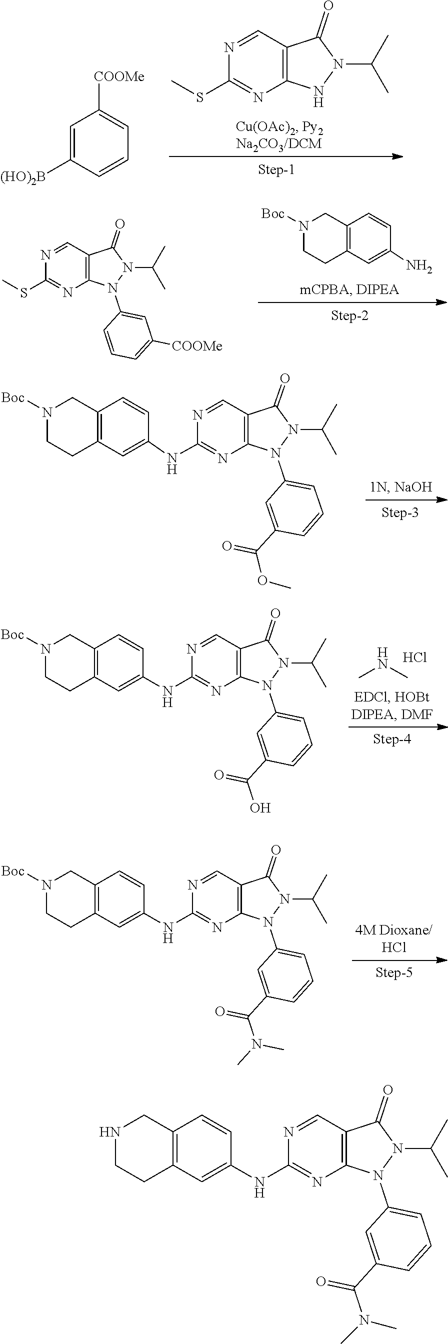

Step-1: Synthesis of methyl 3-(2-isopropyl-6-(methylthio)-3-oxo-2,3-dihydro-1H-pyrazolo[3,4-d]pyrimidin-1-yl)benzoate: To a stirred solution of 2-isopropyl-6-(methylthio)-1,2-dihydro-3H-pyrazolo[3,4-d]pyrimidin-3-one (1.0 g, 4.45 mmol, 1.0 eq) and (3-(methoxycarbonyl)phenyl)boronic acid (1.75 g, 6.68 mmol, 1.5 eq) in DCM (78 mL) was added 2,2-bipyridine (1.04 g, 6.68 mmol, 1.5 eq), copper acetate (1.61 g, 8.90 mmol, 2.0 eq) and Na₂CO₃ (1.41 g, 13.35 mmol, 3.0 eq). The reaction mixture was stirred at RT for 24 h in open air. The progress of reaction was monitored by TLC. After completion, the reaction mixture was filtered over celite to remove inorganic impurities. The filtrate was washed with water, dried over Na₂SO₄, concentrated and purified by column chromatography (Combiflash, Elution: 0-30% EtOAc in hexane) to afford the desired product (450 mg, 22.6%) as an off-white solid. LCMS: 359.2 [M+1]⁺.

Step-2: Synthesis of tert-butyl 6-((2-isopropyl-1-(3-(methoxycarbonyl)phenyl)-3-oxo-2,3-dihydro-1H-pyrazolo[3,4-d]pyrimidin-6-yl)amino)-3,4-dihydroisoquinoline-2(1H)-carboxylate: To a stirring solution of methyl 3-(2-isopropyl-6-(methylthio)-3-oxo-2,3-dihydro-1H-pyrazolo[3,4-d]pyrimidin-1-yl)benzoate (225 mg, 0.63 mmol, 1.0 eq) in toluene (3.0 mL) was added mCPBA (216 mg, 1.25 mmol, 2.0 eq) and allowed to stir at RT for 1 h. Tert-butyl 6-amino-3,4-dihydroisoquinoline-2(1H)-carboxylate (156 mg, 0.63 mmol, 1.0 eq) and DIPEA (0.43 mL, 2.50 mmol, 4.0 eq) were added and allowed to stir at RT for overnight. After completion of reaction, the reaction mixture was diluted with water and extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (260 mg, 74.1%) as brown solid. LCMS: 559.3 [M+1]⁺.

Step-3: Synthesis of 3-(6-((2-(tert-butoxycarbonyl)-1,2,3,4-tetrahydroisoquinolin-6-yl)amino)-2-isopropyl-3-oxo-2,3-dihydro-1H-pyrazolo[3,4-d]pyrimidin-1-yl)benzoic acid: To a solution of tert-butyl 6-((2-isopropyl-1-(3-(methoxycarbonyl)phenyl)-3-oxo-2,3-dihydro-1H-pyrazolo[3,4-d]pyrimidin-6-yl)amino)-3,4-dihydroisoquinoline-2(1H)-carboxylate (260 mg, 0.46 mmol, 1.0 eq) in dioxane (5 mL) and methanol (5 mL) was added 1N NaOH (5 mL) and the reaction mixture was stirred at RT for 24 h. The reaction mixture was concentrated, acidified with 1N HCl and extracted with EtOAc (100 mL×2). The combined organic layer was washed with water (50 mL) and brine solution (50 mL), dried over anhydrous sodium sulfate and concentrated under reduced pressure to get the desired compound (127 mg, 50.1%) as an off-white solid. LCMS: 545.3 [M+1]⁺.

Step-4: Synthesis of tert-butyl 6-((1-(3-(dimethylcarbamoyl)phenyl)-2-isopropyl-3-oxo-2,3-dihydro-1H-pyrazolo[3,4-d]pyrimidin-6-yl)amino)-3,4-dihydroisoquinoline-2(1H)-carboxylate: To a stirred solution of 3-(6-((2-(tert-butoxycarbonyl)-1,2,3,4-tetrahydroisoquinolin-6-yl)amino)-2-isopropyl-3-oxo-2,3-dihydro-1H-pyrazolo[3,4-d]pyrimidin-1-yl)benzoic acid (127 mg, 0.23 mmol, 1.0 eq) and dimethylamine hydrochloride (37 mg, 0.46 mmol, 2.0 eq) in DMF (3 mL) was added EDC. HCl (65 mg, 0.34 mmol, 1.5 eq), HOBt (46 mg, 0.34 mmol, 1.5 eq) and DIPEA (0.15 mL, 0.91 mmol, 4.0 eq). The reaction mixture was stirred at RT for 24 h. After completion, the reaction mixture was diluted with water extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (45 mg, 33.7%) as an off-white solid. LCMS: 572.3 [M+1]⁺.

Step-5: Synthesis of 3-(2-isopropyl-3-oxo-6-((1,2,3,4-tetrahydroisoquinolin-6-yl)amino)-2,3-dihydro-1H-pyrazolo[3,4-d]pyrimidin-1-yl)-N,N-dimethylbenzamide: Tert-butyl 6-((1-(3-(dimethylcarbamoyl)phenyl)-2-isopropyl-3-oxo-2,3-dihydro-1H-pyrazolo[3,4-d]pyrimidin-6-yl)amino)-3,4-dihydroisoquinoline-2(1H)-carboxylate (45 mg, 0.07 mmol, 1.0 eq) was dissolved in dioxane (1 mL), followed by dropwise addition of 4.0 M-HCl in dioxane (1 mL) and allowed to stir at RT for 1 h. After completion of reaction, the reaction mixture was filtered and dried under reduced pressure to afford the desired compound (3 mg, 8.1%) as an off-white solid. LCMS: 472.4 [M+1]⁺; ¹H NMR (400 MHz, DMSO-d₆, Free base): δ 10.13 (s, 1H) 8.79 (s, 1H) 7.62 (d, J=4.8 Hz, 1H) 7.53 (s, 1H) 7.40-7.48 (m, 1H) 7.37 (d, J=8.7 Hz, 1H) 6.86 (d, J=8.7 Hz, 1H) 4.03-4.11 (m, 1H) 3.76 (br s, 2H) 3.00 (br s, 2H) 2.91 (br s, 6H) 2.58 (br s, 2H) 1.17-1.36 (m, 6H).

### Example S-4: Synthesis of 3-(2-isopropyl-3-oxo-6-((1,2,3,4-tetrahydroisoquinolin-7-yl)amino)-2,3-dihydro-1H-pyrazolo[3,4-d]pyrimidin-1-yl)-N,N-dimethylbenzamide (Compound No. 1.4)

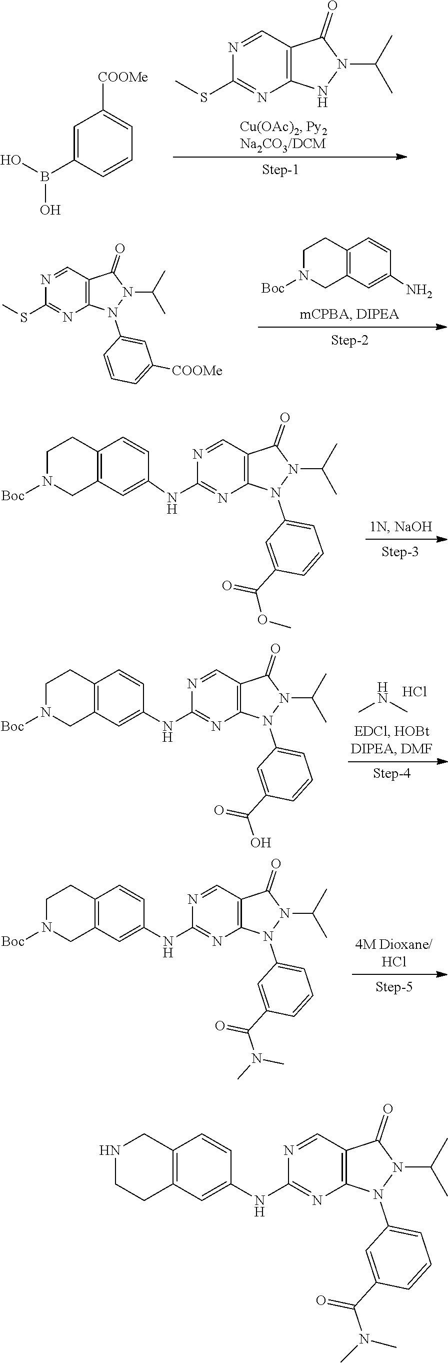

Step-1: Synthesis of methyl 3-(2-isopropyl-6-(methylthio)-3-oxo-2,3-dihydro-1H-pyrazolo[3,4-d]pyrimidin-1-yl)benzoate: To a stirred solution of 2-isopropyl-6-(methylthio)-1,2-dihydro-3H-pyrazolo[3,4-d]pyrimidin-3-one (1.0 g, 4.45 mmol, 1.0 eq) and (3-(methoxycarbonyl)phenyl)boronic acid (1.75 g, 6.68 mmol, 1.5 eq) in DCM (78 mL) was added 2,2-bipyridine (1.04 g, 6.68 mmol, 1.5 eq), copper acetate (1.61 g, 8.90 mmol, 2 eq) and Na₂CO₃ (1.41 g, 13.35 mmol, 3 eq). The reaction mixture was stirred at RT for 24 h in open air. The progress of reaction was monitored by TLC. After completion, the reaction mixture was filtered over celite to remove inorganic impurities. The filtrate was washed with water, dried over Na₂SO₄, concentrated and purified by column chromatography (Combiflash, Elution: 0-30% EtOAc in hexane) to afford the desired product (450 mg, 22.6%) as an off-white solid. LCMS: 359.2 [M+1]⁺.

Step-2: Synthesis of tert-butyl 7-((2-isopropyl-1-(3-(methoxycarbonyl)phenyl)-3-oxo-2,3-dihydro-1H-pyrazolo[3,4-d]pyrimidin-6-yl)amino)-3,4-dihydroisoquinoline-2(1H)-carboxylate: To a stirring solution of methyl 3-(2-isopropyl-6-(methylthio)-3-oxo-2,3-dihydro-1H-pyrazolo[3,4-d]pyrimidin-1-yl)benzoate (225 mg, 0.63 mmol, 1.0 eq) in toluene (3.0 mL) was added mCPBA (216 mg, 1.25 mmol, 2.0 eq) and allowed to stir at RT for 1 h. Tert-butyl 7-amino-3,4-dihydroisoquinoline-2(1H)-carboxylate (156 mg, 0.63 mmol, 1.0 eq) and DIPEA (0.43 mL, 2.50 mmol, 4.0 eq) were added and allowed to stir at RT overnight. After completion of the reaction, the reaction mixture was diluted with water and extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (247 mg, 70.4%) as brown solid. LCMS: 559.3 [M+1]⁺.

Step-3: Synthesis of 3-(6-((2-(tert-butoxycarbonyl)-1,2,3,4-tetrahydroisoquinolin-7-yl)amino)-2-isopropyl-3-oxo-2,3-dihydro-1H-pyrazolo[3,4-d]pyrimidin-1-yl)benzoic acid: To a solution of tert-butyl 7-((2-isopropyl-1-(3-(methoxycarbonyl)phenyl)-3-oxo-2,3-dihydro-1H-pyrazolo[3,4-d]pyrimidin-6-yl)amino)-3,4-dihydroisoquinoline-2(1H)-carboxylate (247 mg, 0.44 mmol, 1.0 eq) in dioxane (5 mL) and methanol (5 mL) was added 1N NaOH (5 mL) and the reaction mixture was stirred at RT for 24 h. The reaction mixture was concentrated, acidified with 1N HCl and extracted with EtOAc (100 mL×2). The combined organic layer was washed with water (50 mL) and brine solution (50 mL), dried over anhydrous sodium sulfate and concentrated under reduced pressure to get the desired compound (117 mg, 48.6%) as an off-white solid. LCMS: 545.3 [M+1]⁺.

Step-4: Synthesis of tert-butyl 7-((1-(3-(dimethylcarbamoyl)phenyl)-2-isopropyl-3-oxo-2,3-dihydro-1H-pyrazolo[3,4-d]pyrimidin-6-yl)amino)-3,4-dihydroisoquinoline-2(1H)-carboxylate: To a stirred solution of 3-(6-((2-(tert-butoxycarbonyl)-1,2,3,4-tetrahydroisoquinolin-7-yl)amino)-2-isopropyl-3-oxo-2,3-dihydro-1H-pyrazolo[3,4-d]pyrimidin-1-yl)benzoic acid (117 mg, 0.21 mmol, 1.0 eq) and dimethylamine hydrochloride (35 mg, 0.43 mmol, 2.0 eq) in DMF (3 mL) was added EDC. HCl (62 mg, 0.32 mmol, 1.5 eq), HOBt (44 mg, 0.32 mmol, 1.5 eq) and DIPEA (0.14 mL, 0.86 mmol, 4.0 eq). The reaction mixture was stirred at RT for 24 h. After completion, the reaction mixture was diluted with water extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (24 mg, 19.5%) as an off-white solid. LCMS: 572.3 [M+1]⁺.

Step-5: Synthesis of 3-(2-isopropyl-3-oxo-6-((1,2,3,4-tetrahydroisoquinolin-7-yl)amino)-2,3-dihydro-1H-pyrazolo[3,4-d]pyrimidin-1-yl)-N,N-dimethylbenzamide: Tert-butyl 7-((1-(3-(dimethylcarbamoyl)phenyl)-2-isopropyl-3-oxo-2,3-dihydro-1H-pyrazolo[3,4-d]pyrimidin-6-yl)amino)-3,4-dihydroisoquinoline-2(1H)-carboxylate (24 mg, 0.04 mmol, 1.0 eq) was dissolved in dioxane (1 mL), followed by dropwise addition of 4.0 M-HCl in dioxane (1 mL) and allowed to stir at RT for 1 h. After completion of reaction, the reaction mixture was filtered and dried under reduced pressure to afford the desired compound (3 mg, 8.1%) as an off-white solid. LCMS: 472.4 [M+1]⁺; ¹H NMR (400 MHz, DMSO-d₆, Free base): δ 10.22 (s, 1H) 8.81 (s, 1H) 7.64 (d, J=4.8 Hz, 2H) 7.53 (s, 1H) 7.36-7.50 (m, 3H) 7.00 (d, J=7.8 Hz, 2H) 4.02-4.12 (m, 2H) 3.92 (br s, 2H) 3.09 (br s, 2H) 3.01 (br s, 3H) 2.91 (br s, 3H) 2.73 (br s, 2H) 1.24-1.34 (m, 6H).

### Example S-5: Synthesis of 1-(2-(tert-butyl)pyridin-4-yl)-2-isopropyl-6-((2-isopropyl-1,2,3,4-tetrahydroisoquinolin-6-yl)amino)-1,2-dihydro-3H-pyrazolo[3,4-d]pyrimidin-3-one (Compound No. 1.5)

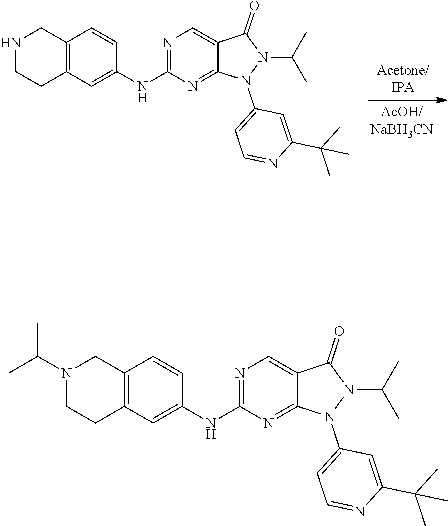

To a stirred solution 1-(2-(tert-butyl)pyridin-4-yl)-2-isopropyl-6-((1,2,3,4-tetrahydroisoquinolin-6-yl)amino)-1,2-dihydro-3H-pyrazolo[3,4-d]pyrimidin-3-one (92 mg, 0.20 mmol, 1.0 eq) and acetone (0.074 mL, 1.0 mmol, 5.0 eq) in IPA (5 mL) was added one drop of acetic acid, followed by addition of sodium cyanoborohydride (32 mg, 0.5 mmol, 2.5 eq) and the reaction mixture was stirred at RT overnight. After completion of reaction, the reaction mixture was diluted with sodium bicarbonate and extracted with EtOAc (50 mL×2). The combined organic layer was washed with water (50 mL) and brine solution (50 mL), dried over anhydrous sodium sulfate and concentrated under reduced pressure to afford crude product, which was purified by preparative chromatography to afford the desired compound (10 mg, 10.0%) as white solid. LCMS: 500.4 [M+1]⁺; ¹H NMR (400 MHz, DMSO-d₆, Formate salt): δ 10.30 (br s, 1H) 8.82 (s, 1H) 8.67 (d, J=5.3 Hz, 1H) 8.33 (br s, 1H) 7.33-7.52 (m, 4H) 7.00 (d, J=8.3 Hz, 1H) 3.89-3.98 (m, 1H) 3.59 (s, 3H) 2.86 (d, J=6.6 Hz, 1H) 2.74-2.82 (m, 2H) 2.58-2.74 (m, 3H) 1.23-1.39 (m, 15H) 1.06 (d, J=6.6 Hz, 6H).

### Example S-6: Synthesis of 4-(2-isopropyl-3-oxo-6-(1,2,3,4-tetrahydroisoquinolin-7-ylamino)-2,3-dihydro-1H-pyrazolo[3,4-d]pyrimidin-1-yl)benzonitrile (Compound No 1.85)

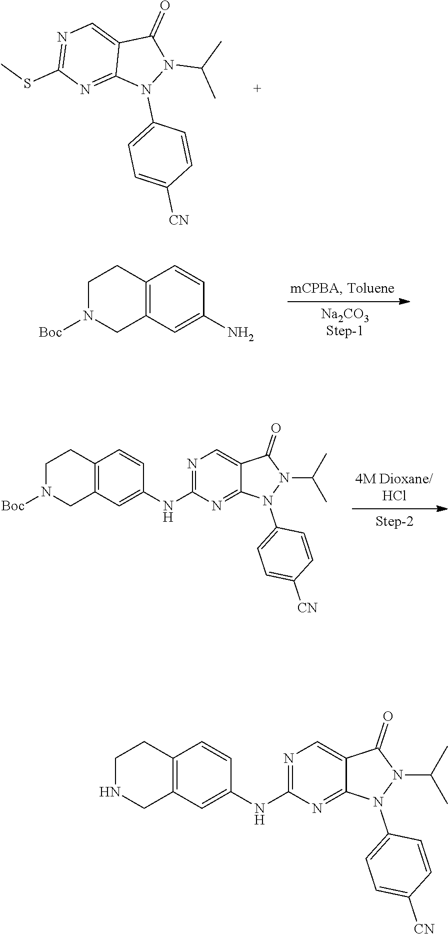

Step-1: Synthesis of tert-butyl 7-(1-(4-cyanophenyl)-2-isopropyl-3-oxo-2,3-dihydro-1H-pyrazolo[3,4-d]pyrimidin-6-ylamino)-3,4-dihydroisoquinoline-2(1H)-carboxylate: To a stirred solution of 4-(2-isopropyl-6-(methylthio)-3-oxo-2,3-dihydro-1H-pyrazolo[3,4-d]pyrimidin-1-yl)benzonitrile (70 mg, 0.21 mmol, 1.0 eq) in toluene (2.0 mL) was added mCPBA (110 mg, 0.43 mmol, 2.0 eq) and allowed to stir at RT for 1 h. Tert-butyl 7-amino-3,4-dihydroisoquinoline-2(1H)-carboxylate (58 mg, 0.24 mmol, 1.1 eq) and Na₂CO₃ (92 mg, 0.86 mmol, 4.0 eq) were added and allowed to stir at RT overnight. After completion of the reaction, the reaction mixture was diluted with water and extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (60 mg, 55.4%) as off-white solid. LCMS: 526.2 (M+1)⁺.

Step-2: Synthesis of 4-(2-isopropyl-3-oxo-6-(1,2,3,4-tetrahydroisoquinolin-7-ylamino)-2,3-dihydro-1H-pyrazolo[3,4-d]pyrimidin-1-yl)benzonitrile: Tert-butyl 7-(1-(4-cyanophenyl)-2-isopropyl-3-oxo-2,3-dihydro-1H-pyrazolo[3,4-d]pyrimidin-6-ylamino)-3,4-dihydroisoquinoline-2(1H)-carboxylate (60 mg, 0.11 mmol, 1.0 eq) was dissolved in dioxane (0.5 mL), followed by dropwise addition of 4.0 M HCl (0.5 mL) and allowed to stir at RT for 1 h. After completion of reaction, the reaction mixture was filtered and purified by reverse phase purification to afford title compound (12 mg, 25.7%) as off-white solid. LCMS: 426.4 (M+1)+; ¹H NMR (400 MHz, DMSO-d₆): δ 10.34 (br s, 1H) 8.83 (s, 1H) 8.28 (s, 1H) 8.05 (m, J=8.3 Hz, 2H) 7.77 (m, J=8.3 Hz, 2H) 7.58 (br s, 1H) 7.38 (d, J=8.7 Hz, 1H) 7.08 (d, J=8.3 Hz, 1H) 3.90-4.05 (m, 3H) 3.10 (br s, 2H) 2.75 (br s, 2H) 1.32 (d, J=6.6 Hz, 6H).

### Example S-7: Synthesis of 4-(2-isopropyl-3-oxo-6-(1,2,3,4-tetrahydroisoquinolin-6-ylamino)-2,3-dihydro-1H-pyrazolo[3,4-d]pyrimidin-1-yl)benzonitrile (Compound No 1.86)

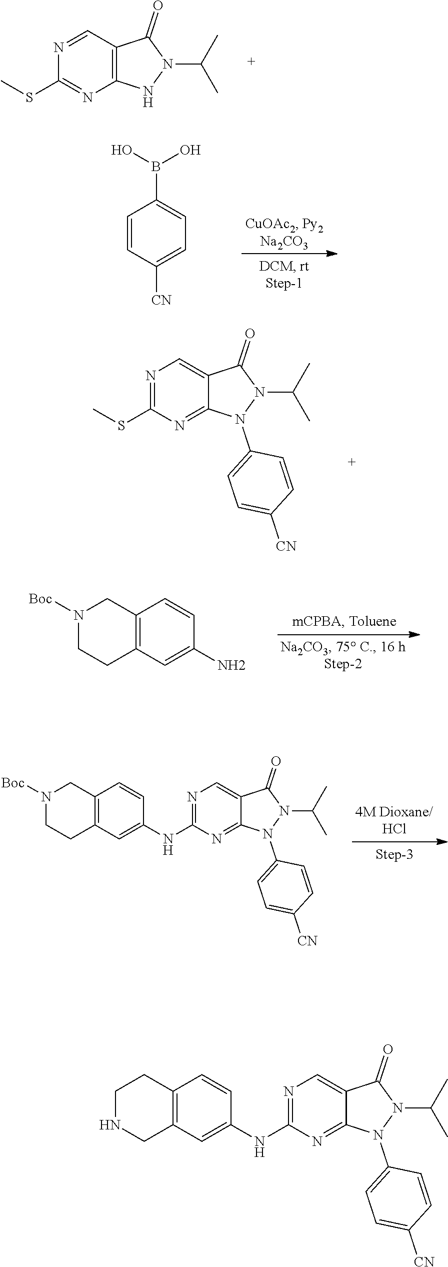 [SMILES: CSc1ncc2c(=O)n(C(C)C)[nH]c2n1 ; N#Cc1ccc(B(O)O)cc1 ; CSc1ncc2c(=O)n(C(C)C)n(-c3ccc(C#N)cc3)c2n1 ; CC(C)(C)OC(=O)N1CCc2cc(N)ccc2C1 ; CC(C)n1c(=O)c2cnc(Nc3ccc4c(c3)CCN(C(=O)OC(C)(C)C)C4)nc2n1-c1ccc(C#N)cc1 ; CC(C)n1c(=O)c2cnc(Nc3ccc4c(c3)CNCC4)nc2n1-c1ccc(C#N)cc1]

Step-1: Synthesis of 4-(2-isopropyl-6-(methylthio)-3-oxo-2,3-dihydro-1H-pyrazolo[3,4-d]pyrimidin-1-yl)benzonitrile: To a stirred solution of 2-isopropyl-6-(methylthio)-1,2-dihydro-3H-pyrazolo[3,4-d]pyrimidin-3-one (224 mg, 1.0 mmol, 1.0 eq) and 4-cyanophenylboronic acid (176 mg, 1.2 mmol, 1.2 eq) in DCM (40 mL) was added 2,2-bipyridine (303 mg, 2.0 mmol, 2.0 eq), copper acetate (364 mg, 2.0 mmol, 2.0 eq) and Na₂CO₃ (318 mg, 3.0 mmol, 3.0 eq). The reaction mixture was stirred at RT for 24 h in open air. The progress of reaction was monitored by TLC. After completion, the reaction mixture was filtered over celite to remove inorganic impurities. The filtrate was washed with water, dried over Na₂SO₄, concentrated and purified by column chromatography (Combiflash, Elution: 0-30% EtOAc in hexane) to afford the desired product (200 mg, 61.5%) as an off-white solid. LCMS: 326.1 (M+1)⁺.

Step-2: Synthesis of tert-butyl 6-(1-(4-cyanophenyl)-2-isopropyl-3-oxo-2,3-dihydro-1H-pyrazolo[3,4-d]pyrimidin-6-ylamino)-3,4-dihydroisoquinoline-2(1H)-carboxylate: To a stirred solution 4-(2-isopropyl-6-(methylthio)-3-oxo-2,3-dihydro-1H-pyrazolo[3,4-d]pyrimidin-1-yl)benzonitrile (70 mg, 0.21 mmol, 1.0 eq) in toluene (2.0 mL) was added mCPBA (110 mg, 0.43 mmol, 2.0 eq) and allowed to stir at RT for 1 h. Tert-butyl 6-amino-3,4-dihydroisoquinoline-2(1H)-carboxylate (58 mg, 0.24 mmol, 1.1 eq) and Na₂CO₃ (92 mg, 0.86 mmol, 4.0 eq) were added and allowed to stir at RT overnight. After completion of reaction, the reaction mixture was diluted with water and extracted with EtOAc (50 mL×2). The combined organic layer was washed with water (50 mL) and brine solution (50 mL), dried over anhydrous sodium sulfate and concentrated under reduced pressure to afford crude product, which was purified by flash chromatography [silica gel 100-200 mesh; elution 0-50% EtOAc in hexane] to afford the desired compound (80 mg, 72.1%) as an off-white solid. LCMS: 526.2 (M+1)⁺.

Step-3: Synthesis of 4-(2-isopropyl-3-oxo-6-(1,2,3,4-tetrahydroisoquinolin-6-ylamino)-2,3-dihydro-1H-pyrazolo[3,4-d]pyrimidin-1-yl)benzonitrile: Tert-butyl 6-(1-(4-cyanophenyl)-2-isopropyl-3-oxo-2,3-dihydro-1H-pyrazolo[3,4-d]pyrimidin-6-ylamino)-3,4-dihydroisoquinoline-2(1H)-carboxylate (80 mg, 0.15 mmol, 1.0 eq) was dissolved in dioxane (1 mL), followed by dropwise addition of 4.0 M-HCl (1 mL) and allowed to stir at RT for 1 h. After completion of reaction, the reaction mixture was filtered and purified by reverse phase purification to afford the title compound (20 mg, 31.8%) as an off-white solid. LCMS: 426.4 (M+1)+; ¹H NMR (400 MHz, DMSO-d₆): δ 10.36 (br s, 1H), 8.85 (s, 1H), 8.05 (m, J=8.3 Hz, 2H), 7.77 (m, J=8.7 Hz, 2H) 7.66 (br s, 1H) 7.42 (d, J=8.7 Hz, 1H) 7.09 (d, J=8.3 Hz, 1H) 4.07 (br s, 2H) 3.98 (dt, J=13.7, 6.9 Hz, 1H) 3.25 (br s, 2H) 2.87 (br s, 2H) 1.32 (d, J=7.0 Hz, 6H).

### Example S-8: Synthesis of 2,2,2-trifluoroacetaldehyde compound with 6-((2-(3-aminopropyl)-1,2,3,4-tetrahydroisoquinolin-6-yl)amino)-1-(2-(tert-butyl)pyridin-4-yl)-2-isopropyl-1,2-dihydro-3H-pyrazolo[3,4-d]pyrimidin-3-one (Compound No. 1.87)

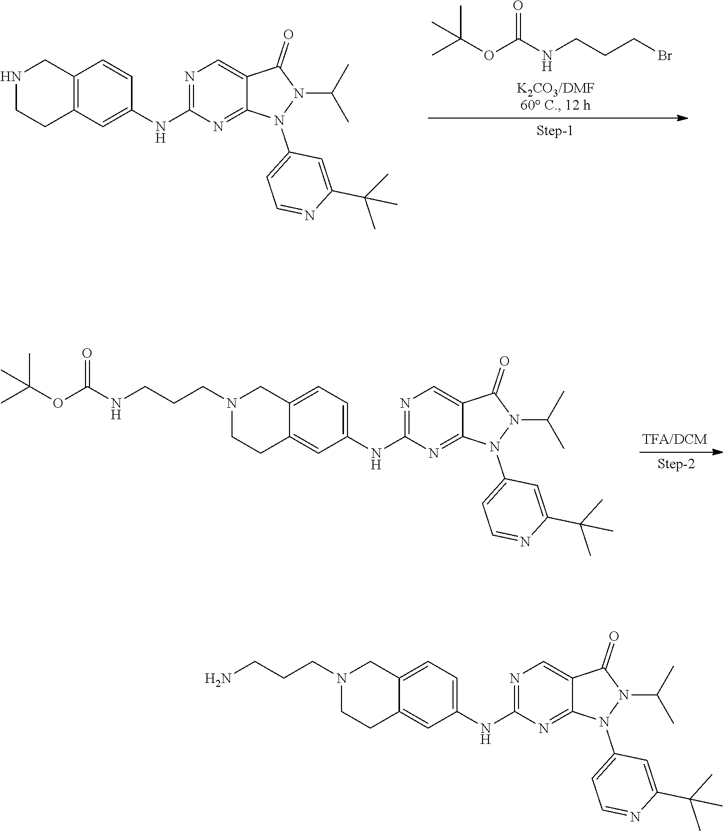 [SMILES: CC(C)n1c(=O)c2cnc(Nc3ccc4c(c3)CCNC4)nc2n1-c1ccnc(C(C)(C)C)c1 ; CC(C)(C)OC(=O)NCCCBr ; CC(C)n1c(=O)c2cnc(Nc3ccc4c(c3)CCN(CCCNC(=O)OC(C)(C)C)C4)nc2n1-c1ccnc(C(C)(C)C)c1 ; CC(C)n1c(=O)c2cnc(Nc3ccc4c(c3)CCN(CCCN)C4)nc2n1-c1ccnc(C(C)(C)C)c1]

Step-1: Synthesis of tert-butyl (3-(6-((1-(2-(tert-butyl)pyridin-4-yl)-2-isopropyl-3-oxo-2,3-dihydro-1H-pyrazolo[3,4-d]pyrimidin-6-yl)amino)-3,4-dihydroisoquinolin-2(1H)-yl)propyl)carbamate: To a stirred solution of 1-(2-(tert-butyl)pyridin-4-yl)-2-isopropyl-6-((1,2,3,4-tetrahydroisoquinolin-6-yl)amino)-1,2-dihydro-3H-pyrazolo[3,4-d]pyrimidin-3-one (590 mg, 1.28 mmol, 1.0 eq) in DMF (12 mL), tert-butyl (3-bromopropyl)carbamate (338 mg, 1.41 mmol, 1.1 eq) was added and the reaction mixture was stirred 60° C. for 12 h. The reaction mixture was poured in ice cold water. The precipitate formed was collected by filtration to afford the desired product (318 mg, 40.1%) as an off-white solid. LCMS: 615.4 (M+1)⁺.

Step-2: Synthesis of 2,2,2-trifluoroacetaldehyde compound with 6-((2-(3-aminopropyl)-1,2,3,4-tetrahydroisoquinolin-6-yl)amino)-1-(2-(tert-butyl)pyridin-4-yl)-2-isopropyl-1,2-dihydro-3H-pyrazolo[3,4-d]pyrimidin-3-one: To a stirred solution of tert-butyl (3-(6-((1-(2-(tert-butyl)pyridin-4-yl)-2-isopropyl-3-oxo-2,3-dihydro-1H-pyrazolo[3,4-d]pyrimidin-6-yl)amino)-3,4-dihydroisoquinolin-2(1H)-yl)propyl)carbamate (300 mg, 0.49 mmol, 1.0 eq) in DCM (20 mL), TFA (6 mL) was added drop wise. The reaction mixture was stirred at RT for 2 h. The reaction mixture was concentrated and triturate was diethyl ether. The precipitate formed was collected by filtration to get the desired product (300 mg, 76.0%) as an off-white solid. LCMS: 515.6 [M+1]⁺; ¹H NMR (400 MHz, DMSO-d₆): δ10.49 (br s, 1H) 10.11 (br s, 1H) 8.88 (s, 1H) 8.72 (d, J=5.2 Hz, 1H) 7.89 (br s, 2H) 7.73 (br s, 1H) 7.60 (d, J=5.7 Hz, 2H) 7.41 (s, 1H) 7.18 (d, J=8.3 Hz, 1H) 4.51 (d, J=14.4 Hz, 2H) 4.27 (br s, 2H) 3.97 (dt, J=13.2, 6.7 Hz, 1H) 3.38-3.30 (br s, 2H) 3.16-3.11 (br s, 2H) 2.93 (d, J=7.0 Hz, 2H) 1.96-2.11 (m, 2H) 1.29-1.44 (m, 15H).

The compounds disclosed therein are prepared according to the experimental details exemplified in Examples S1-S8 and Scheme 1 to Scheme 5, using the appropriate starting materials and reagents.

### BIOLOGICAL EXAMPLES

### Example B1. WEE1 IC₅₀ Determination

IC₅₀ values of compounds against WEE1 kinase enzyme were determined by LanthaScreen™ Terbium Labeled TR-FRET assay. Kinase assays were performed in 1× kinase buffer (#PV6135, Invitrogen, Life Technologies Grand Island, N.Y.) where total reaction volume was 10 μL in low-volume 384-well plates (#4511, Corning). Serially diluted compounds (3-fold) were incubated with WEE1 Enzyme (1 nM) (#PR7373A, Invitrogen, Life Technologies, Grand Island, N.Y.) for 10 min, and a mixture of ATP (10 μM) (#A1852, Sigma, St. Louis, Mo.) and fluorescent-PolyGT substrate (200 nM) (#PV3610, Invitrogen, Life Technologies, Grand Island, N.Y.) was added and incubated in the dark at room temperature for 1 h. After 1 h, a 10 μL stop solution containing terbium labeled antibody (4 nM) (#PV3529, Invitrogen, Life Technologies Grand Island, N.Y.) and EDTA (#E5134, Sigma, St. Louis, Mo.) (20 mM) in TR-FRET dilution buffer (#PV3574, Invitrogen, Life Technologies, Grand Island, N.Y.) was added. Readings were taken in a Synergy Neo Plate reader (BioTek, Winooski, Vt.) at single excitation of 340 nm and dual emission at 495 nm and 520 nm respectively.

The % activity of test samples was calculated as (Sample−Min)*100/(Max−Min). [Max: DMSO control, complete reaction with enzyme & DMSO and Min: No enzyme & DMSO]. Percent inhibition (100−% activity) was fitted to the “four-parameter logistic model” in XLfit for determination of IC₅₀ values. The results are shown in Table 2.

TABLE 2

| Compound No. | Wee1 IC₅₀ (μM) |
|---|---|
| 1.1 | 0.107 |
| 1.2 | 0.004 |
| 1.3 | 0.002 |
| 1.4 | 0.003 |
| 1.5 | 0.021 |
| 1.85 | 0.005 |
| 1.86 | 0.008 |
| 1.87 | 0.004 |

### Example B2. PKMYT1 IC₅₀ Determination

Inhibition of PKMYT1 kinase activity by test compounds is measured by the HotSpot Kinase Assay at Reaction Biology Corporation (Malvern, Pa.). Briefly, Myelin Basic Protein substrate is prepared in reaction buffer (20 mM Hepes (pH 7.5), 10 mM MgCl₂, 1 mM EGTA, 0.01% Brij35, 0.02 mg/mL BSA, 0.1 mM Na₃VO₄, 2 mM DTT, 1% DMSO). PKMYT1 kinase is delivered into the substrate solution and gently mixed. Test compounds in 100% DMSO are added into the kinase reaction mixture by Acoustic technology (Echo550; nanoliter range) and incubated for 20 min at room temperature. ³³P-ATP is delivered into the reaction mixture to initiate the reaction. Reactions are carried out at 10 μM ATP. After a 2 hour incubation at room temperature, kinase activity is detected by P81 filter-binding method. Compounds are tested in 10-dose IC₅₀ mode with a 3-fold serial dilution. A nonlinear regression model with a sigmoidal dose response and variable slope within GraphPad Prism (GraphPad Software, San Diego, Calif.) is used to calculate the IC₅₀ value of individual test compounds.

### Example B3. Determination of Potency of Compounds in Cytotoxicity Assay in A427 Cell Line

A427 (HTB-53; ATCC), a lung epithelial cell line, was seeded in medium (MEM, 41090101; Gibco) at a cell count of 1500 cells per 100 μL per well in a 96 well edge plate (167425; ThermoFisher). Cells were allowed to grow at 37° C. for 24 hr in 5% CO₂ environment (culture conditions) in a Nuaire incubator (humidified). Serially diluted test compounds (100 μL) within the desired testing concentration ranges were added to the culture plate and the cells were further incubated in culture conditions for 72 hr. The experiment was terminated at the designated incubation time by replacing the medium with 100 μL of 1 mM of resazurin (R7017; Sigma) prepared in culture medium, and the plates were further incubated in culture conditions for 4-6 hr. Fluorescence was recorded using a multimodal plate reader (Biotek Synergy Neo) at an excitation wavelength of 535 nm and emission wavelength of 590 nm to obtain relative fluorescence units. Data were analysed as follows: the background fluorescence (blank containing only medium) value was subtracted from each reading and normalized with the vehicle control (DMSO treated cells) to obtain percent survival/proliferation. Percent survival was subtracted from 100 to get the percent inhibition of proliferation which was used to calculate IC₅₀ values. Potency of compounds in other cell lines (such as A549, As-Pc-1, Panc 10.05, A172, U-87 MG) may be determined in an analogous manner. The results are shown in Table 4.

TABLE 3

| Compound No. | A427 IC₅₀ (μM) |
|---|---|
| 1.1 | >30 |
| 1.2 | 0.295 |
| 1.3 | 8.162 |
| 1.4 | 3.509 |
| 1.5 | 2.069 |
| 1.85 | 1.865 |
| 1.86 | 1.066 |

### Example B4. Determination of Potency of Compounds in Cell Proliferation Assay in Selected Cancer Cell Lines and Cellular PD Effects

The effects of test compounds are studied in additional cell lines with various histotypes, such as LoVo colorectal adenocarcinoma, NCI-H460 large-cell lung carcinoma, HCT-116 colorectal carcinoma, and A2780 ovarian cancer cells. The cancer cells are harvested during the logarithmic growth period and counted. Cell concentrations are adjusted to the appropriate number with suitable medium, and 90 μL cell suspensions are added to 96-well plates. After cells are seeded, the plates are shaken gently to distribute cells evenly and incubated at 37° C., 5% CO₂ on day 1.

Cells are treated with test compounds at 9 concentrations within a desired concentration range (e.g. 1.5 nM-10 μM) on day 2 by series diluting the test compound stock solution (10 mM in DMSO) with culture medium. Cell viability is assessed by Cell Titer-Glo® as recommended by Promega (Cat. No.: G7572, Promega) typically 72 h post-treatment.

Cell viability data are plotted using GraphPad Prism (version 5, GraphPad Software, Inc., San Diego, Calif.). In addition, a nonlinear regression model with a sigmoidal dose response and variable slope within GraphPad Prism is used to calculate the IC₅₀ value of individual test compounds.

Test compounds may be studied in the same and/or other cancer cell lines with varying sensitivities to reported Wee1 inhibiting compounds using similar proliferation methods with possible variations in cell seeding densities and/or incubation durations.

### Example B5. Determination of Potency of Compounds by Assay of Cellular PD Effects

pCDC₂ and γ-H2AX are two clinically relevant biomarkers associated with Wee1 inhibition. CDC₂Y15 phosphorylation in cells was reported to be abolished by Wee1 inhibitors (Gavory G et. al., Almac Discovery, AACR poster, 2016). γ-H2AX, a DNA double-strand break marker, was upregulated by Wee1 treatment in Wee1 sensitive cell lines (Guertin A D et al., Molecular Cancer Therapeutics, 2013). The effects of selected test compounds on pCDC₂ and γ-H2AX are assessed in selected cancer cell lines post 24 or 48 hr treatment using Western blotting methods with selective antibodies (Guertin A D et al., Molecular Cancer Therapeutics, 2013).

Changes in the levels of phospho-CDC₂ following treatment of cells with test compounds were assessed by enzyme-linked immunosorbent assay (ELISA). A427 cells were plated in 6-well plates and cultured for 24 hr to approximately 80-90% confluency. Medium was replaced, and the cells were treated with the vehicle control or the test compound at several different concentrations. After incubation of treated cells in cell culture conditions for a specified time (e.g., 24 hr), cells were rinsed with ice-cold PBS and lysed in 1× cell lysis buffer containing protease inhibitors and phosphatase inhibitors. The cells were scraped from the plate with a cell scraper after a brief incubation on ice and transferred to a centrifuge tube, and then subjected to three freeze-thaw cycles in liquid nitrogen and a 37° C. water bath for further lysis. The lysates were centrifuged to pellet cell debris (using, for example, a 10 min centrifugation of 2000×g at 4° C.) and the supernatants transferred to fresh tubes on ice. The protein concentrations of the samples were estimated by the Bradford method or equivalent. The ELISA was carried out with the PathScan® Phospho-CDC₂ (Tyr15) Sandwich ELISA Kit (Cat. #7176, Cell Signaling Technology, Danvers, Mass.) according to the manufacturer's instructions. Results are shown in Table 4.

TABLE 4

| Compound No. | A427 phospho-CDC2 IC₅₀ (μM) |
|---|---|
| 1.85 | 0.278 |

Changes in the levels of phospho-CDC₂ are alternatively or additionally analyzed by Western blotting of the samples using a primary antibody to phospho-CDC₂ such as phospho-CDC₂ (Tyr15) (10A11) rabbit mAb (Cat. #4539, Cell Signaling Technology) or rabbit polyclonal anti-CDK1 (phospho Y15) antibody (Cat. #ab47594, Abcam, Cambridge, United Kingdom).

### Example B6. Evaluation of Test Compound in Mouse Xenograft Models

To examine the in vivo antitumor activity of test compound (as a single agent and in combination with other agents such as gemcitabine, nab-paclitaxel and temozomide), tumor growth experiments are performed in a cell line xenograft model and/or a PDX model. The cell line is chosen based on the in vitro studies described above. The PDX model to be used is established from a tumor taken directly from a patient with, for example, pancreatic ductal adenocarcinoma (PDAC) or glioblastoma.

Cells or tumor chucks are implanted subcutaneously into the flanks of nude mice and allowed to grow until the tumor size reaches 200 mm³. Tumors are measured using a caliper and tumor volumes calculated using the formula: Tumor volume=(a×b²/2) where ‘b’ is the smallest diameter and ‘a’ is the largest diameter. Once the established tumors reach approximately 200 mm³, the mice are then stratified into treatment groups. The treatment groups are, for example: vehicle control, gemcitabine+nab-paclitaxel, test compound alone, gemcitabine+nab-paclitaxel+test compound at 10 mice per group. The treatment groups are alternatively, for example: vehicle control, temozolomide, test compound alone, temozolomide+test compound. The exact treatment groups, drug dose, and dosing schedule are determined specifically for each study according to standard practice. Tumor growth is monitored, and volume recorded at regular intervals. When the individual tumor of each mouse reaches an approximate end-point (tumor volume>1,500 mm³), the mouse is sacrificed with regulated CO₂. The tumor growth inhibition (TGI) is calculated by comparing the control group's tumor measurements with the other study groups once the predetermined endpoint is reached in the control group. Alternatively, cells are implanted orthotopically and overall survival is measured.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

What is claimed is:

1. A compound of Formula (I): 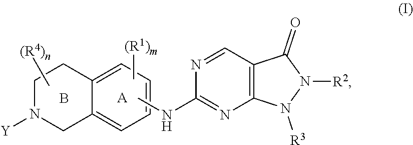
or a salt thereof, wherein:
Y is hydrogen or R⁴;
m is 0, 1, 2, or 3;
n is 0, 1, 2, 3, or 4;
R¹ is independently F, Cl, or methyl;
R² is independently C₁-C₆ alkyl, C₃-C₆ cycloalkyl or —(C₁-C₃ alkylene)CF₃;
R³ is 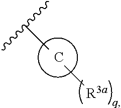
wherein:
ring C is a 5- or 6-membered heteroaryl or phenyl,
each R^{3a} is independently halogen, C₃-C₆ cycloalkyl, or —(C₁-C₆ alkylene)R′, wherein the C₁-C₆ alkylene is optionally substituted by oxo and wherein R′ is hydrogen, —CN, —NR¹⁹R²⁰, C₁-C₆ alkoxy, or —OH,
q is 0, 1, 2, 3, or 4;
each R⁴ is independently oxo, C₁-C₆ alkyl, C₂-C₆ alkenyl, C₂-C₆ alkynyl, halogen, —C(O)R⁷, —C(O)OR¹⁷, —C(O)NR¹⁷R¹⁸, —CN, —Si(C₁-C₆ alkyl)₃, —OR¹⁷, —NR¹⁷R¹⁸, —OC(O)NR¹⁷R¹⁸, —NR¹⁷C(O)R¹⁸, —S(O)₂R¹⁷, —NR¹⁷S(O)₂R¹⁸, —S(O)₂NR¹⁷R¹⁸, C₃-C₆ cycloalkyl, 3- to 6-membered heterocyclyl, —(C₁-C₃ alkylene)CN, —(C₁-C₃ alkylene)OR¹⁷, —(C₁-C₃ alkylene)NR¹⁷R¹⁸, —(C₁-C₃ alkylene)CF₃, —(C₁-C₃ alkylene)C(O)R¹⁷, —(C₁-C₃ alkylene)C(O)NR¹⁷R¹⁸, —(C₁-C₃ alkylene)NR¹⁷C(O)R¹⁸, —(C₁-C₃ alkylene)S(O)₂R¹⁷, —(C₁-C₃ alkylene)NR¹⁷S(O)₂R⁸, —(C₁-C₃ alkylene)S(O)₂NR¹⁷R¹⁸, —(C₁-C₃ alkylene)(C₃-C₆ cycloalkyl) or —(C₁-C₃ alkylene)(3- to 6-membered heterocyclyl), wherein each R⁴ is independently optionally substituted by halogen, oxo, —OR¹⁹, —NR¹⁹R²⁰, or —C(O)R¹⁹,
or two R⁴, when bound to the same carbon, are taken together with the carbon to which they are attached to form a C₃-C₆ cycloalkyl or 3- to 6-membered heterocyclyl, each is optionally substituted by R¹⁹; and
each R¹⁷, R¹⁸, R¹⁹, and R²⁰ is independently hydrogen, C₃-C₆ cycloalkyl, 3-6 membered heterocyclyl or C₁-C₆ alkyl, each of which is optionally substituted by halogen, oxo or —OH,
or R¹⁷ and R¹⁸ are taken together with the atom to which they attached to form a 3-6 membered heterocyclyl optionally substituted by halogen, oxo or —OH.

2. The compound of claim 1, or a salt thereof, wherein the compound is of Formula (II):

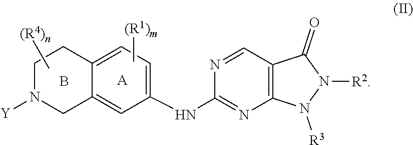

3. The compound of claim 1, or a salt thereof, wherein the compound is of Formula (III):

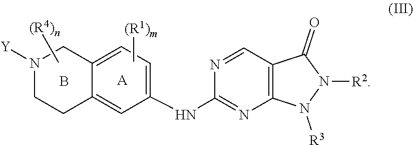

4. The compound of any one of claims 1-3, or a salt thereof, wherein R² is C₁-C₆ alkyl.

5. The compound of claim 4, or a salt thereof, wherein R² is isopropyl or ethyl.

6. The compound of any one of claims 1-3, or a salt thereof, wherein R² is C₃-C₆ cycloalkyl.

7. The compound of claim 6, or a salt thereof, wherein R² is cyclopropyl.

8. The compound of any one of claims 1-3, or a salt thereof, wherein R² is —(C₁-C₃ alkylene)CF₃.

9. The compound of claim 8, or a salt thereof, wherein R² is —CH₂CF₃.

10. The compound of any one of claims 1-9, or a salt thereof, wherein ring C is selected from the group consisting of:

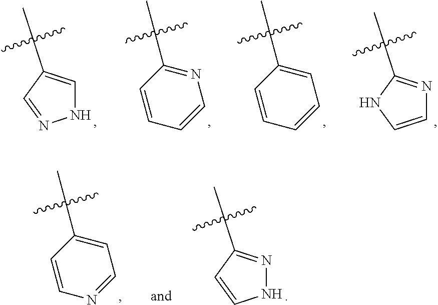 [SMILES: *c1cn[nH]c1 ; *c1ccccn1 ; *c1ccccc1 ; *c1ncc[nH]1 ; *c1ccncc1 ; *c1cc[nH]n1]

11. The compound of any one of claims 1-10, or a salt thereof, wherein R^{3a} is C₃-C₆ cycloalkyl.

12. The compound of any one of claims 1-10, or a salt thereof, wherein R^{3a} is —(C₁-C₆alkylene)R′, wherein the C₁-C₆ alkylene is optionally substituted by oxo and wherein R′ is hydrogen, —CN, —NR¹⁹R²⁰, C₁-C₆ alkoxy, or —OH.

13. The compound of claim 12, or a salt thereof, wherein R′ is hydrogen.

14. The compound of claim 12, or a salt thereof, wherein R′ is —CN.

15. The compound of claim 12, or a salt thereof, wherein R′ is —NR¹⁹R²⁰.

16. The compound of claim 12, or a salt thereof, wherein R′ is C₁-C₆ alkoxy.

17. The compound of claim 12, or a salt thereof, wherein R′ is —OH.

18. The compound of any one of claims 1-10, or a salt thereof, wherein R^{3a} is selected from the group consisting of: 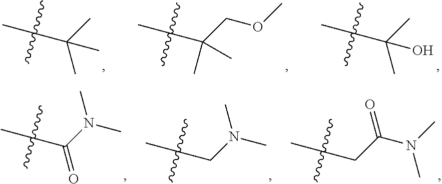
fluoro, and cyclopropyl.

19. The compound of any one of claims 1-18, or a salt thereof, wherein q is 1.

20. The compound of any one of claims 1-9, or a salt thereof, wherein R³ is selected from the group consisting of:

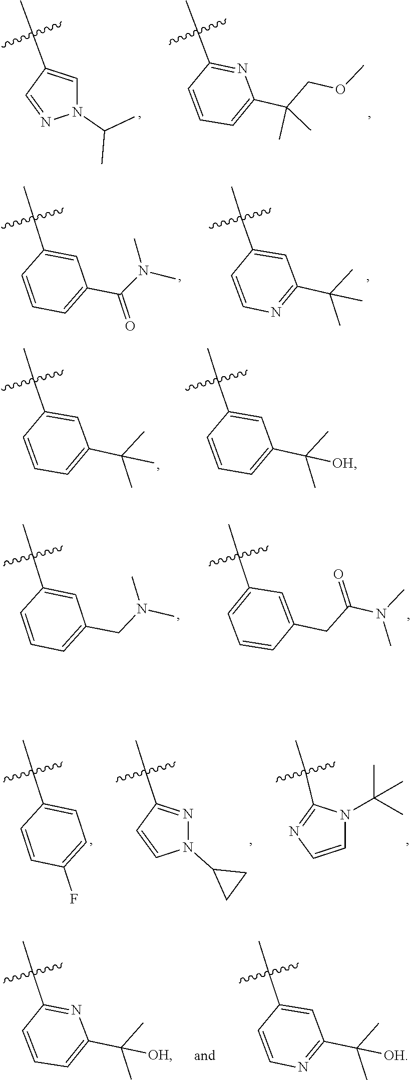

21. The compound of any one of claims 1-20, or a salt thereof, wherein m is 0.

22. The compound of any one of claims 1-20, or a salt thereof, wherein m is 1 and Rn is F.

23. The compound of any one of claims 1-22, or a salt thereof, wherein Y is hydrogen.

24. The compound of any one of claims 1-23, or a salt thereof, wherein Y is C₁-C₆ alkyl.

25. The compound of any one of claims 1-24, or a salt thereof, wherein n is 0.

26. The compound of any one of claims 1-24, or a salt thereof, wherein n is 1.

27. The compound of any one of claims 1-24, or a salt thereof, wherein n is 2.

28. The compound of any one of claims 1-27, or a salt thereof, wherein each R⁴ is independently C₁-C₆ alkyl, or two R⁴, when bound to the same carbon are taken together with the carbon or carbons to which they are attached to form a C₃-C₆ cycloalkyl.

29. The compound of any one of claims 1-20, or a salt thereof, wherein ring A, ring B, R¹, and R⁴ are taken together to form a moiety selected from the group consisting of:

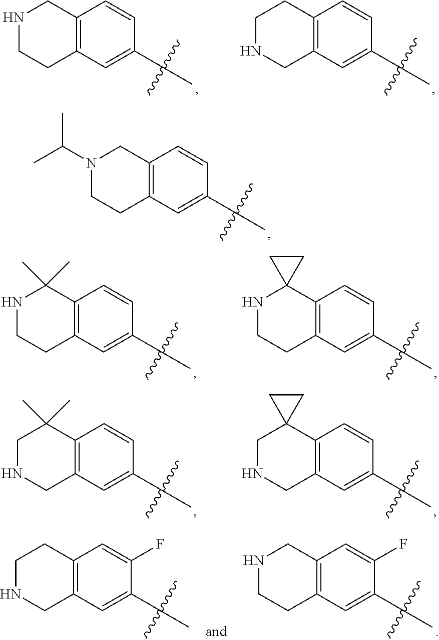

30. A compound or a salt thereof, wherein the compound is selected from the group consisting of compounds in Table 1.

31. The compound of claim 30, wherein the compound is a pharmaceutically acceptable salt of a compound in Table 1.

32. A pharmaceutical composition comprising a compound of any one of claims 1-30, or a pharmaceutically acceptable salt thereof, and a pharmaceutically acceptable carrier.

33. A method of treating a cancer in an individual in need thereof comprising administering to the individual a therapeutically effective amount of a compound of any one of claims 1-30, or a salt thereof.

34. The method of claim 33, further comprising administering a radiation therapy to the individual.

35. The method of claim 33 or 34, further comprising administering to the individual a therapeutically effective amount of an additional therapeutic agent.

36. The method of claim 35, wherein the additional therapeutic agent is a cancer immunotherapy agent or a chemotherapeutic agent.

37. The method of claim 35 or 36, wherein the additional therapeutic agent is a DNA alkylating agent, a platinum-based chemotherapeutic agent, a kinase inhibitor or a DNA damage repair (DDR) pathway inhibitor.

38. The method of any one of claims 33-37, wherein the cancer comprises a mutant TP53 gene.

39. The method of any one of claims 33-38, comprising selecting the individual for treatment based on (i) the presence of one or more mutations in the TP53 gene in the cancer, or (ii) expression of mutant p53 in the cancer.

40. A method of suppressing a G₂-M checkpoint in a cell, comprising administering a compound of any one of claims 1-30, or a salt thereof, to the cell.

41. A method of inducing premature mitosis in a cell, comprising administering a compound of any one of claims 1-30, or a salt thereof, to the cell.

42. A method of inducing apoptosis in a cell, comprising administering a compound of any one of claims 1-30, or a salt thereof, to the cell.

43. A method of inhibiting Wee1 in a cell, comprising administering a compound of any one of claims 1-30, or a salt thereof, to the cell.

44. A method of inhibiting Wee1, comprising contacting Wee1 with a compound of any one of claims 1-30, or a salt thereof.

45. The method of claim 44, wherein the inhibitor binds to Wee1 with an IC₅₀ of less than 1 μM according to a kinase assay.

46. Use of a compound of any one of claims 1-30, or a salt thereof, in the manufacture of a medicament for treatment of cancer.

47. A kit comprising a compound of any one of claims 1-30, or a salt thereof.
